(19) United States
(12) Patent Application Publication
(10) Pub. No.: US 2019/0320900 A1
(43) Pub. Date: Oct. 24, 2019

(54) TELEMEDICINE SYSTEM

(71) Applicant: CureCompanion Inc., North Brunswick, NJ (US)

(72) Inventor: Mukul Majmudar, Piscataway, NJ (US)

(21) Appl. No.: 15/956,199

(22) Filed: Apr. 18, 2018

(51) Int. Cl. A61B 5/00; G16H 40/67

(52) U.S. Cl. CPC A61B 5/0022; G16H 10/60; G16H 40/67

(57) ABSTRACT

Various example embodiments generally relate to a remote healthcare capability for supporting remote healthcare services for a patient by a healthcare professional. The remote healthcare capability may be provided within the context of a healthcare services system. The remote healthcare capability may enable a patient located remotely from a healthcare professional (e.g., at home, in a nursing home, in an emergency medical facility, in a rural location, or the like) to nevertheless receive healthcare services from the healthcare professional. The remote healthcare capability may include a telemedicine platform configured to support integration of telemedicine applications (e.g., a patient application for patients and a healthcare professional application for healthcare professionals) and telemedicine devices (e.g., an integrated patient care kit, such as a bag, a cart, or the like) in order to support various remote healthcare scenarios in which a patient located remotely from a healthcare professional is able to receive healthcare services from the healthcare professional.

### TECHNICAL FIELD

Various example embodiments relate generally to healthcare and, more particularly but not exclusively, to telemedicine-based healthcare.

### BACKGROUND

Various technological advances have fostered the development of various telemedicine capabilities; however, while telemedicine capabilities continue to be developed, use of telemedicine by healthcare professionals and patients still has not become widespread.

Improvements in telemedicine capabilities may spur wider adoption of telemedicine by healthcare professionals and patients, thereby providing various benefits for healthcare professionals and patients.

### SUMMARY

Various example embodiments relate generally to telemedicine capabilities that are configured to support use of telemedicine by healthcare professionals and patients.

In at least some example embodiments, an apparatus is provided. The apparatus includes at least one processor. The apparatus includes at least one memory including computer program code. The at least one memory and the computer program code are configured to, with the at least one processor, cause the apparatus to at least receive, from a patient application running on a patient user device, a request to schedule a video appointment with a healthcare professional, send, toward a healthcare professional application running on a healthcare professional user device of the healthcare professional, an indication of the request to schedule the video appointment with the healthcare professional, receive, from the healthcare professional application, an indication of an acceptance of the request to schedule the video appointment with the healthcare professional, facilitate a video call between the healthcare professional user device and the patient user device, receive, from a telemedicine device, medical examination information associated with the patient, send, toward the healthcare professional application, the medical examination information associated with the patient, send, toward the healthcare professional application, additional medical information associated with the patient, and receive, from the healthcare professional application, information related to diagnosis of the patient by the healthcare professional.

In at least some example embodiments, a method is provided. The method includes receiving, from a patient application running on a patient user device, a request to schedule a video appointment with a healthcare professional. The method includes sending, toward a healthcare professional application running on a healthcare professional user device of the healthcare professional, an indication of the request to schedule the video appointment with the healthcare professional. The method includes receiving, from the healthcare professional application, an indication of an acceptance of the request to schedule the video appointment with the healthcare professional. The method includes facilitating a video call between the healthcare professional user device and the patient user device. The method includes receiving, from a telemedicine device, medical examination information associated with the patient. The method includes sending, toward the healthcare professional application, the medical examination information associated with the patient. The method includes sending, toward the healthcare professional application, additional medical information associated with the patient. The method includes receiving, from the healthcare professional application, information related to diagnosis of the patient by the healthcare professional.

In at least some example embodiments, a non-transitory computer readable medium is provided. The non-transitory computer-readable medium includes program instructions for causing an apparatus to at least receive, from a patient application running on a patient user device, a request to schedule a video appointment with a healthcare professional, send, toward a healthcare professional application running on a healthcare professional user device of the healthcare professional, an indication of the request to schedule the video appointment with the healthcare professional, receive, from the healthcare professional application, an indication of an acceptance of the request to schedule the video appointment with the healthcare professional, facilitate a video call between the healthcare professional user device and the patient user device, receive, from a telemedicine device, medical examination information associated with the patient, send, toward the healthcare professional application, the medical examination information associated with the patient, send, toward the healthcare professional application, additional medical information associated with the patient, and receive, from the healthcare professional application, information related to diagnosis of the patient by the healthcare professional.

In at least some example embodiments, an apparatus is provided. The apparatus includes means for receiving, from a patient application running on a patient user device, a request to schedule a video appointment with a healthcare professional. The apparatus includes means for sending, toward a healthcare professional application running on a healthcare professional user device of the healthcare professional, an indication of the request to schedule the video appointment with the healthcare professional. The apparatus includes means for receiving, from the healthcare professional application, an indication of an acceptance of the request to schedule the video appointment with the healthcare professional. The apparatus includes means for facilitating a video call between the healthcare professional user device and the patient user device. The apparatus includes means for receiving, from a telemedicine device, medical examination information associated with the patient. The apparatus includes means for sending, toward the healthcare professional application, the medical examination information associated with the patient. The apparatus includes means for sending, toward the healthcare professional application, additional medical information associated with the patient. The apparatus includes means for receiving, from the healthcare professional application, information related to diagnosis of the patient by the healthcare professional.

In at least some embodiments, a patient care unit is provided. The patient care unit includes a portable housing. The portable housing includes a first portion including a computing device having a touch-sensitive display, a microphone, a speaker, and a set of communication interfaces. The portable housing includes a second portion, configured to provide a base for the portable housing, including a set of receptacles configured to house a set of medical instruments, wherein the set of medical instruments includes a set of wired medical instruments and a set of wireless medical instruments. The portable housing includes a hinge pivotally connecting the first portion to the second portion to permit the first portion to pivot with respect to the second portion between a closed position and an open position. The portable housing includes a wired medical instrument interface control element communicatively connected to the computing device. The wired medical instrument interface control element is configured to control interfacing between the wired medical instruments and the computing device. The set of communication interfaces includes a local wireless communication interface configured to support interfacing between the wireless medical instruments and the computing device.

### BRIEF DESCRIPTION OF THE DRAWINGS

The teachings herein can be readily understood by considering the following detailed description in conjunction with the accompanying drawings, in which:

FIG. 1 depicts an example of a healthcare services system configured to support remote healthcare for a patient by a healthcare professional using a telemedicine platform;

FIG. 2 depicts an example of a telemedicine bag configured for use by a patient or a healthcare provider to enable the patient to obtain remote healthcare services from a healthcare professional;

FIG. 3 depicts an example of a telemedicine cart configured for use by a patient or a healthcare provider to enable the patient to obtain remote healthcare services from a healthcare professional;

FIG. 4 depicts an example of a telemedicine server configured to support a telemedicine platform configured to enable patients to obtain remote healthcare services from healthcare professionals;

FIGS. 5A-5F depict examples of graphical user interfaces of a patient application configured for use by a patient to obtain remote healthcare services from a healthcare professional;

FIGS. 6A-6D depict examples of graphical user interfaces of a healthcare professional application configured for use by a healthcare professional to provide remote healthcare services to a patient;

FIG. 7 depicts an example embodiment of a method for use by an integrated telemedicine platform for supporting providing of remote healthcare services to a patient;

FIG. 8 depicts an example embodiment of a method for interaction by a user device with a patient care unit for supporting providing of remote healthcare services to a patient;

FIG. 9 depicts an example embodiment of a method for interactions by a patient care unit for supporting providing of remote healthcare services to a patient;

FIG. 10 depicts an example embodiment of method configured for use by a patient application at a user device of a patient;

FIG. 11 depicts an example embodiment of method configured for use by a healthcare professional application at a user device of a healthcare professional; and

FIG. 12 depicts a high-level block diagram of a computer suitable for use in performing various functions presented herein.

To facilitate understanding, identical reference numerals have been used, where possible, to designate identical elements that are common to the figures.

### DETAILED DESCRIPTION

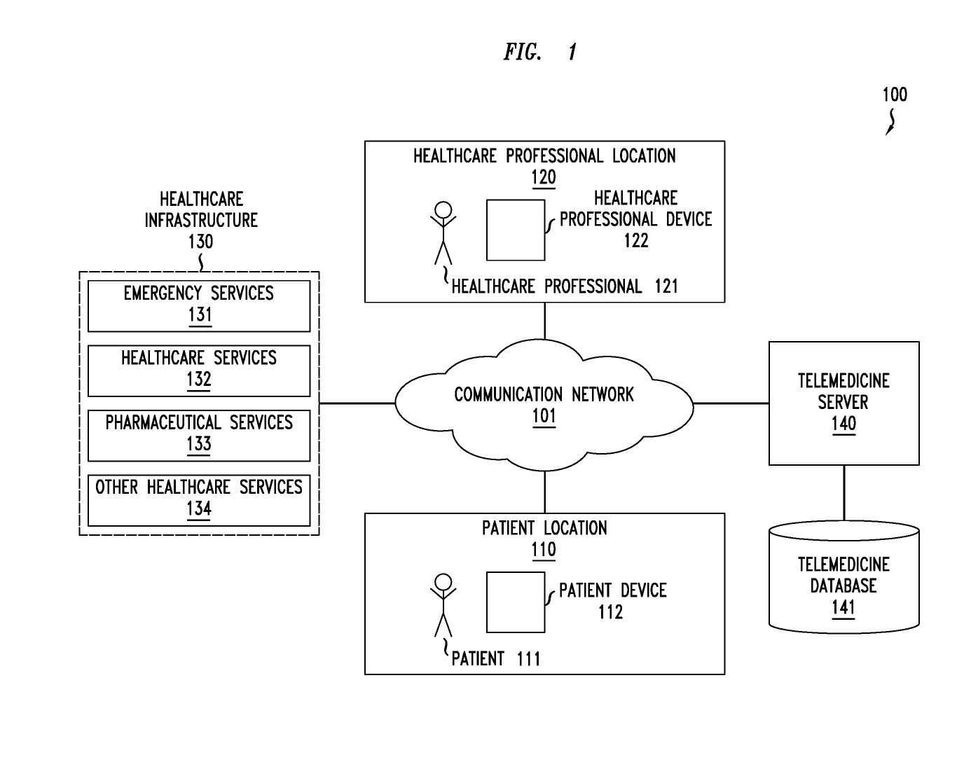

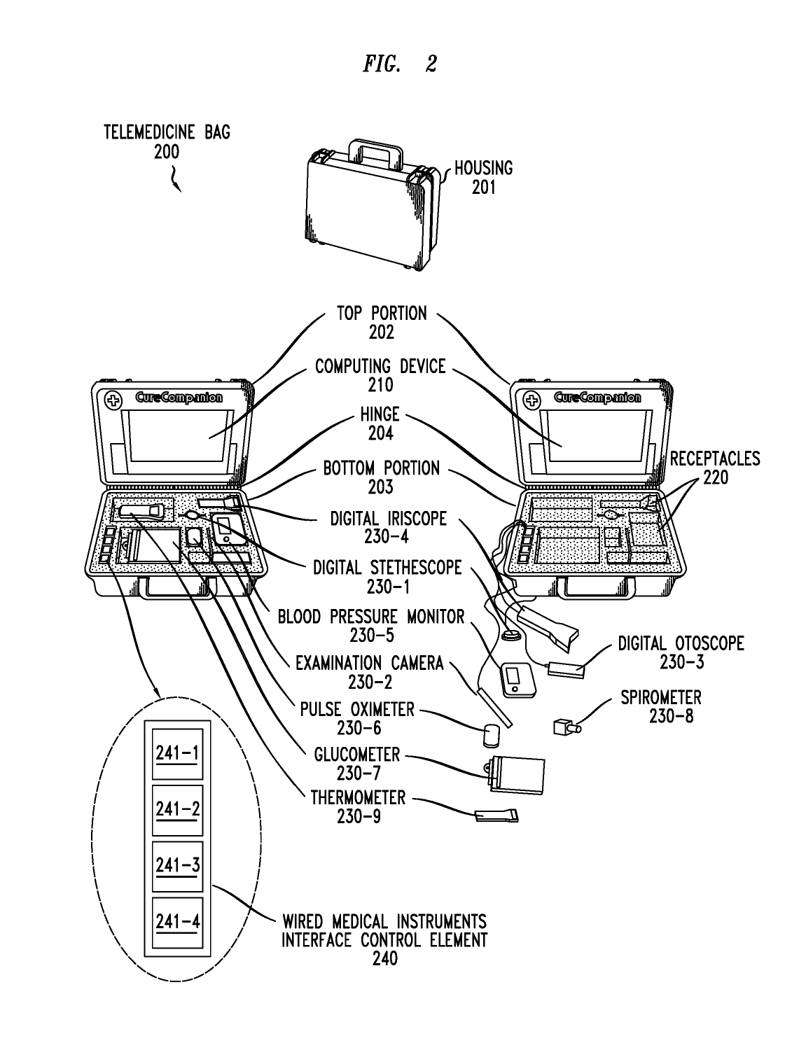

The present disclosure generally discloses a remote healthcare capability for supporting remote healthcare services for a patient by a healthcare professional. The remote healthcare capability may be provided within the context of a healthcare services system. The remote healthcare capability may enable a patient located remotely (e.g., at home, in a nursing home, in an emergency medical facility, in a rural location, or the like) from a healthcare professional to nevertheless receive healthcare services from the healthcare professional. The remote healthcare capability may include a telemedicine device (e.g., an integrated patient care kit, such as a bag, a cart, or the like) which may be used by a patient located remotely from a healthcare professional (or a user —such as a healthcare professional, family member, or the like—on behalf of the patient) to receive healthcare services from the healthcare professional. The remote healthcare capability may include a patient application or webpage which may be used by a patient located remotely from a healthcare professional to receive healthcare services from the healthcare professional, which may be used by a healthcare professional co-located with the patient to facilitate providing of healthcare services to the patient by a healthcare professional located remotely from the patient, or the like, as well as various combinations thereof. The remote healthcare capability may include a healthcare professional application or webpage which may be used by a healthcare professional located remotely from a patient to provide healthcare services to the patient, which may be used by a healthcare professional co-located with the patient to facilitate providing of healthcare services to the patient by a healthcare professional located remotely from the patient, or the like, as well as various combinations thereof. The remote healthcare capability may include a telemedicine platform configured to support integration of the applications (e.g., the patient application and the healthcare professional application) and the telemedicine devices in order to support various remote healthcare scenarios. The remote healthcare capability may be configured to provide a comprehensive telemedicine platform and patient care unit supporting effective remote patient care, providing a telemedicine platform suitable for a variety of remote care scenarios and providing an integrated patient care device (e.g., a bag, a cart, or the like) configured to enable a healthcare professional to remotely perform a detailed physical examination to gain a precise, definitive picture of the condition of the patient. The remote healthcare capability may be configured to provide an integrated telemedicine platform that is configured to create unique remote healthcare facilities. The remote healthcare capability may include various other capabilities which may enable interfacing between a patient and a healthcare professional such that the healthcare professional can provide healthcare services to the patient remotely. It will be appreciated that these and various other embodiments and advantages and potential advantages of the remote healthcare capability may be further understood by way of reference to the example healthcare services system of FIG. 1.

FIG. 1 depicts an example of a healthcare services system configured to support remote healthcare for a patient by a healthcare professional using a telemedicine platform.

The healthcare services system 100 includes a communication network 101, a patient location 110 at which a patient 111 is located, a healthcare professional location 120 at which a healthcare professional 121 is located, a healthcare infrastructure 130, and a telemedicine server 140.

The communication network 101 may be any communication network suitable for supporting communications within the healthcare services system 100. For example, the communication network 101 may include one or more wireline networks (e.g., one or more wireless networks, or the like, as well as various combinations thereof.

The patient location 110 may be any location at which the patient 111 may be located. The patient location 110 is expected to be remote from the healthcare professional location 120. The patient location 110 may be the home of the patient 111, the office of the patient 111, a location to which the patient 111 has traveled for work, a location to which the patient 111 has traveled for vacation, a location to which the patient 111 has traveled for care (e.g., a skilled nursing care facility, an urgent care facility, a rural clinic, or the like), or the like. The patient location 110 may be too remote to permit the patient 111 to travel to the healthcare professional location 120 (although it will be appreciated that there may be other reasons that the patient 111 may not travel to the healthcare profession location 120, such as where the patient is too sick, does not have the means to travel there, would prefer to remain at their current location, or the like, as well as various combinations thereof).

The patient 111 has a patient device 112 by which the patient 111 at the patient location 110 may interact with the healthcare professional 121 at the healthcare professional location 120.

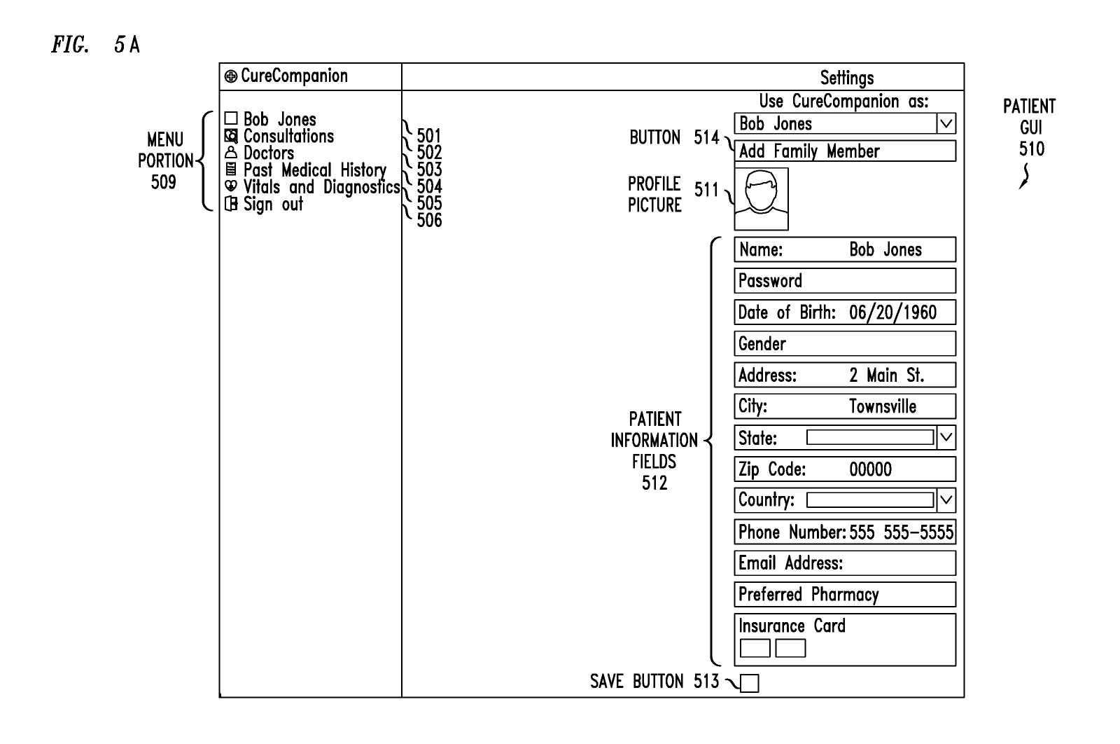

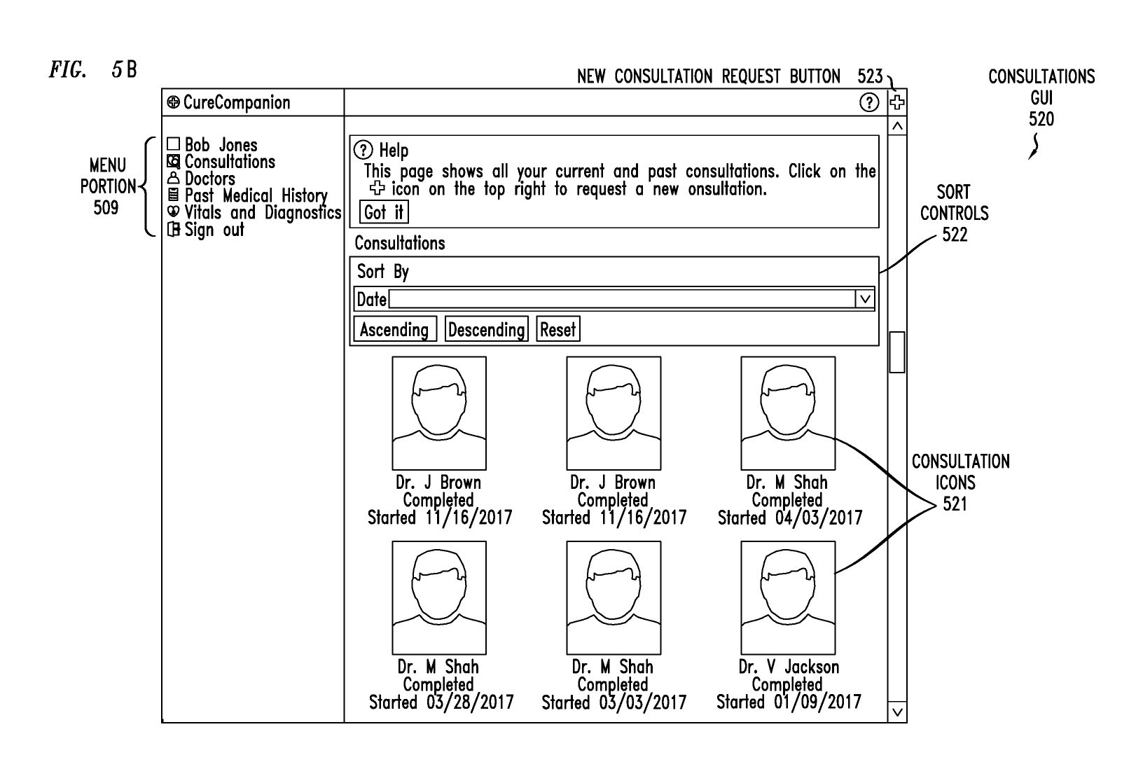

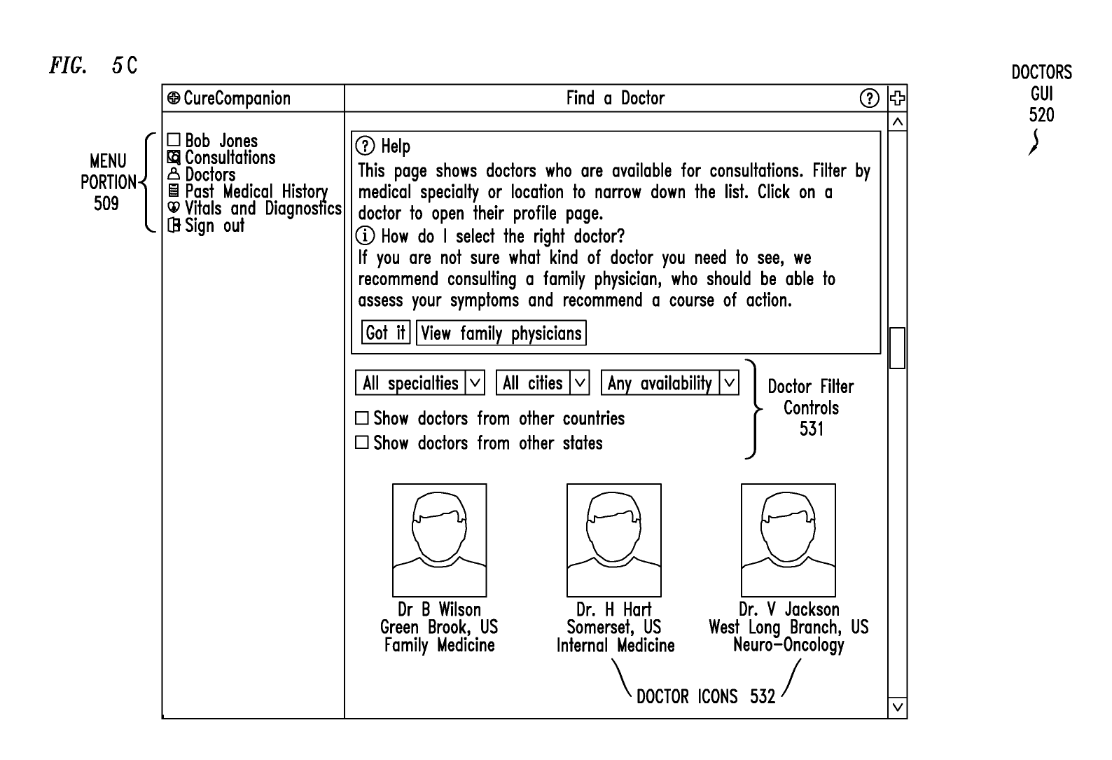

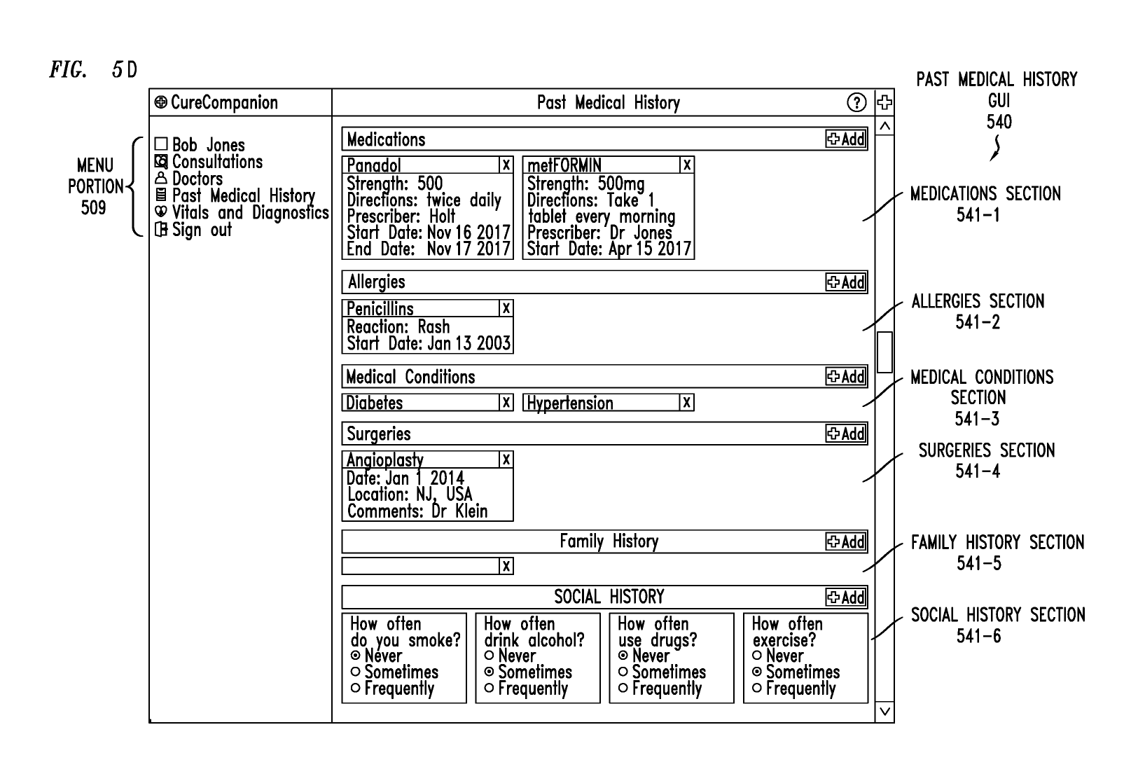

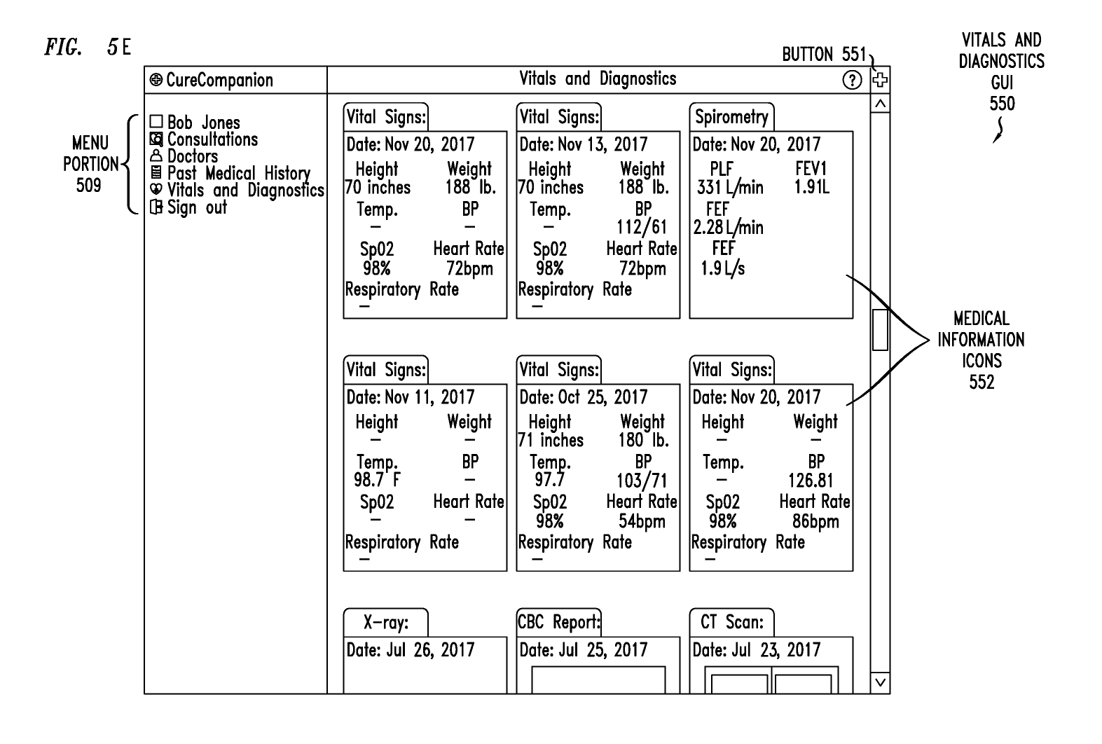

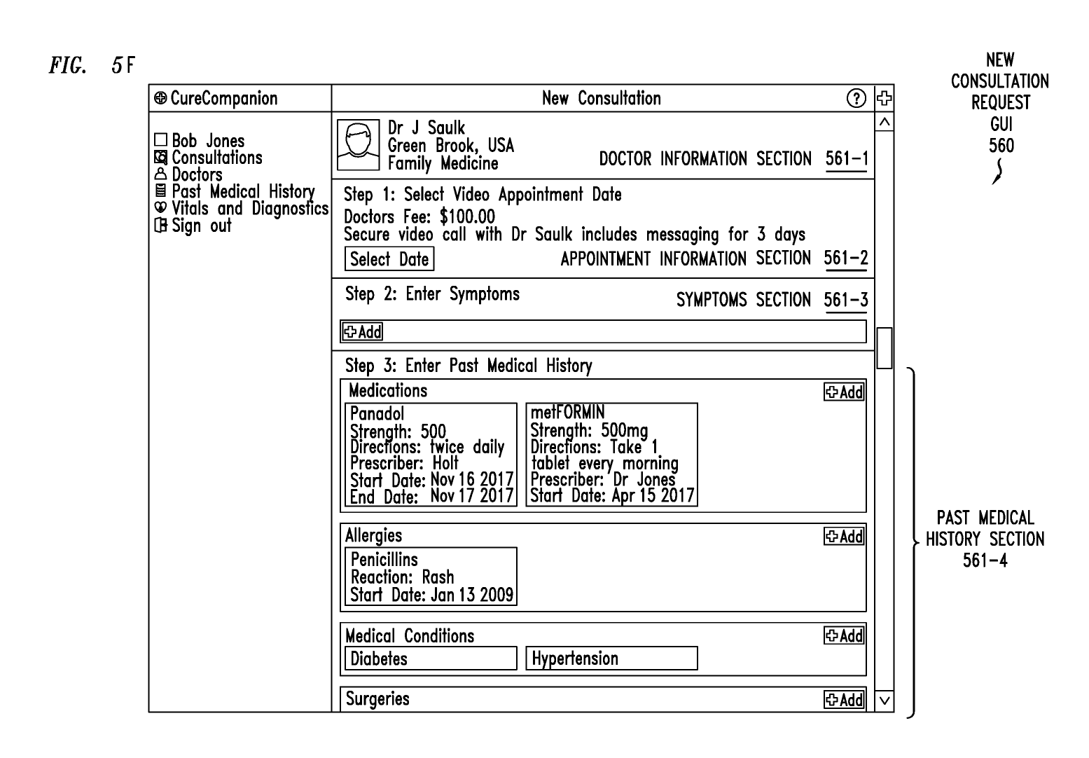

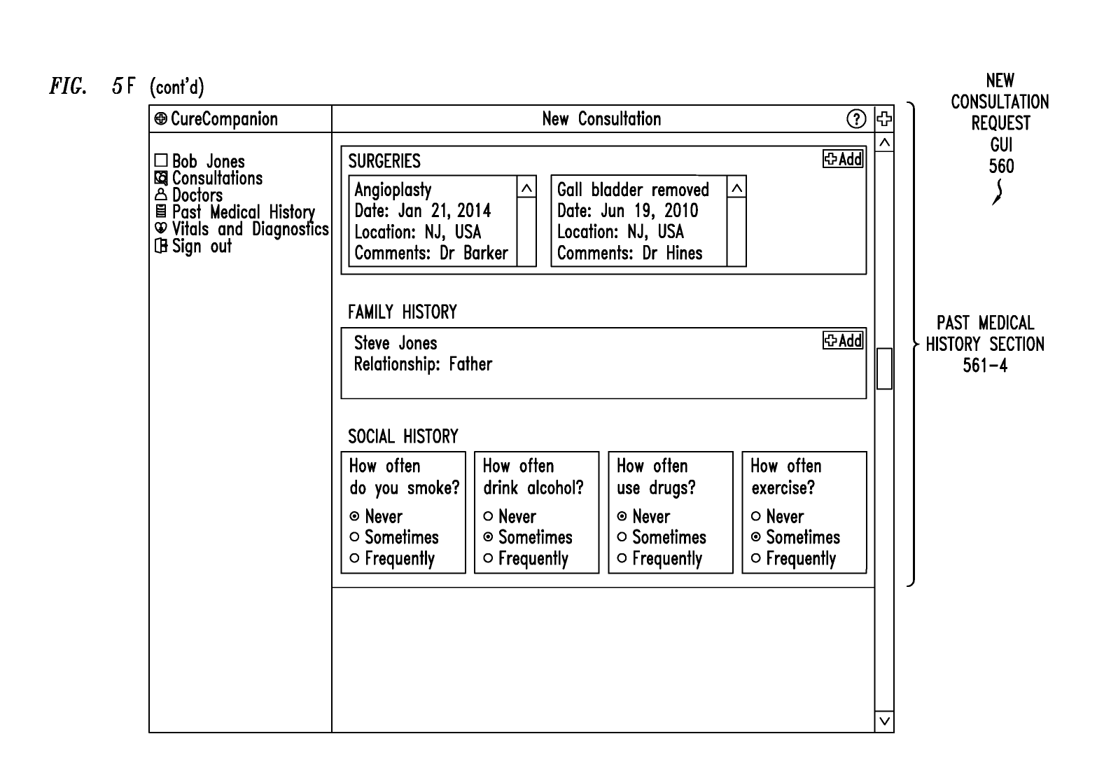

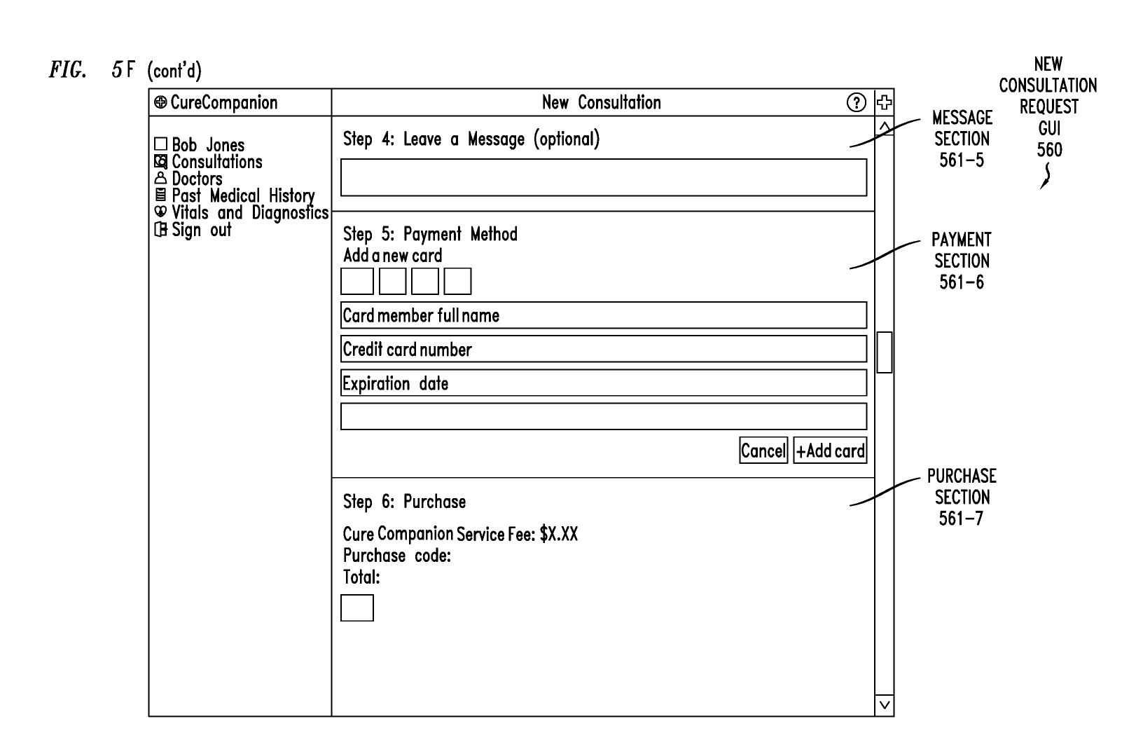

In at least some embodiments, the patient device 112 may be a communication device of the patient 111 (e.g., a smartphone, a computer, or the like). In at least some such embodiments, the patient device 112 may be running an application installed on the patient device 112 (e.g., a CureCompanion for Patients application, as presented with respect to FIGS. 5A-5F, or other similar application).

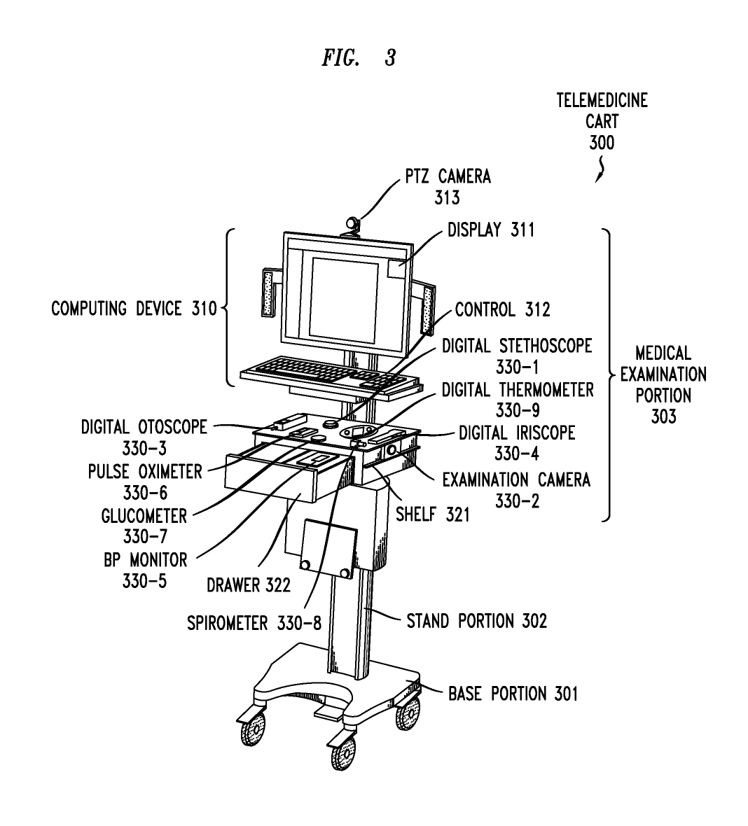

In at least some embodiments, the patient device 112 may be a telemedicine device. In at least some such embodiments, the telemedicine device may be a CureCompanion telemedicine bag as presented with respect to FIG. 2, a CureCompanion telemedicine cart as presented with respect to FIG. 3, or other similar telemedicine device configured to enable healthcare services to be provided to the patient 111 by the healthcare professional 121). The telemedicine device may include an assortment of devices (e.g., user interaction devices, communication devices, medical instruments, connectivity devices, and so forth) configured to work seamlessly with the telemedicine platform provided by the telemedicine server 140.

It will be appreciated that, although primarily presented with respect to an embodiment in which the patient 111 uses a single patient device 112 at the patient location 110 to obtain healthcare services from the health professional 121, the patient 111 may use multiple patient devices 112 at the patient location 110 to obtain healthcare services from the health professional 121 (e.g., multiple communication devices, a communication device and a telemedicine kit, various IoT devices which may be configured to support remote health monitoring for the patient 111 (e.g., heart rate monitors, blood pressure monitors, blood sugar monitors, blood oxygen monitors, temperature monitors, or the like, as well as various combinations thereof), or the like, as well as various combinations thereof. It will be appreciated that, although primarily presented with respect to a single patient location 110, the healthcare services system 100 may support multiple patient locations (and potentially a large number of patient locations).

The healthcare professional location 120 may be any location at which the healthcare professional 121 may be located. The healthcare professional location 120 is expected to be remote from the patient location 110. The healthcare professional location 120 may be the home of the healthcare professional 121, the office of the healthcare professional 121, a location to which the healthcare professional 121 has traveled for work (e.g., a hospital, a conference, or the like) or vacation, or the like. The healthcare professional location 120 may be a call center to which the healthcare professional 121 may travel in order to provide remote healthcare services for the patient 111 and potentially other patients (omitted for purposes of clarity), a call center specifically staffed with the healthcare professional 121 and potentially other healthcare professionals (omitted for purposes of clarity) to provide remote healthcare services for patients such as patient 111, or the like. The healthcare professional location 120 may be too remote to permit the patient 111 to travel to the healthcare professional location 120 (although it will be appreciated that there may be other reasons that the patient 111 may not travel to the healthcare profession location 120, such as where the patient is too sick, does not have the means to travel there, would prefer to remain at their current location, or the like, as well as various combinations thereof).

The healthcare professional 121 has a healthcare professional device 122 by which the healthcare professional 121 at the healthcare professional location 120 may interact with the patient 111 at the patient location 110.

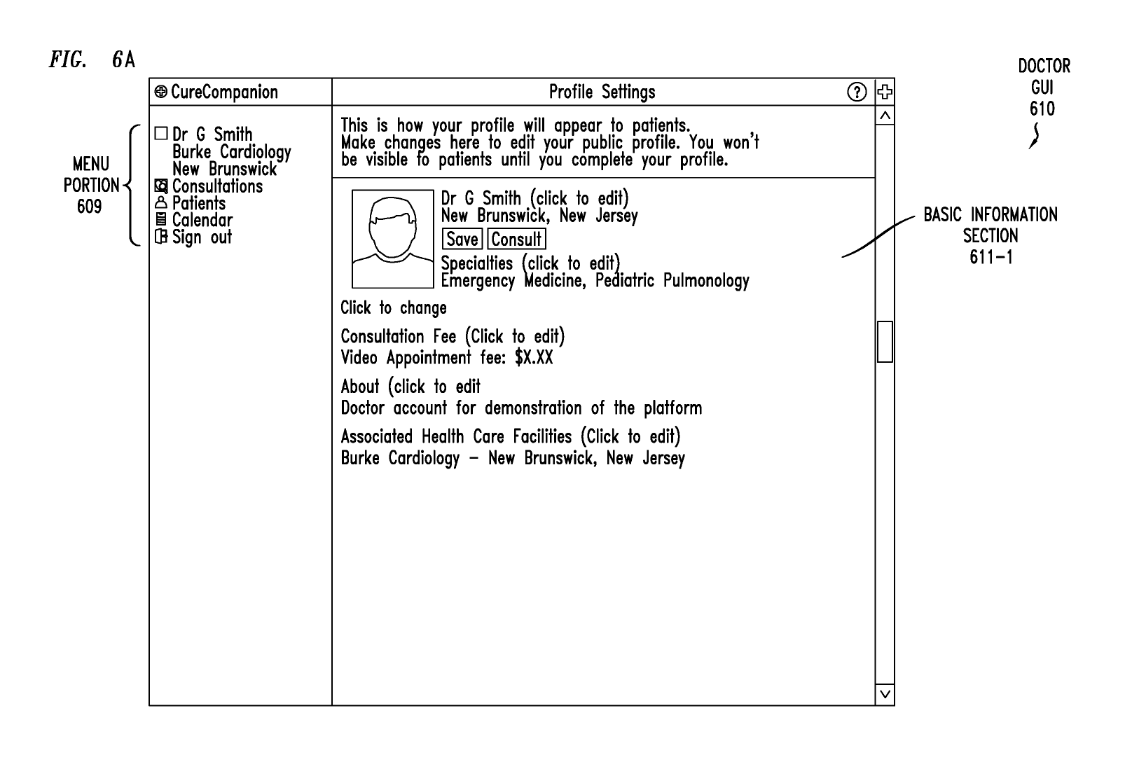

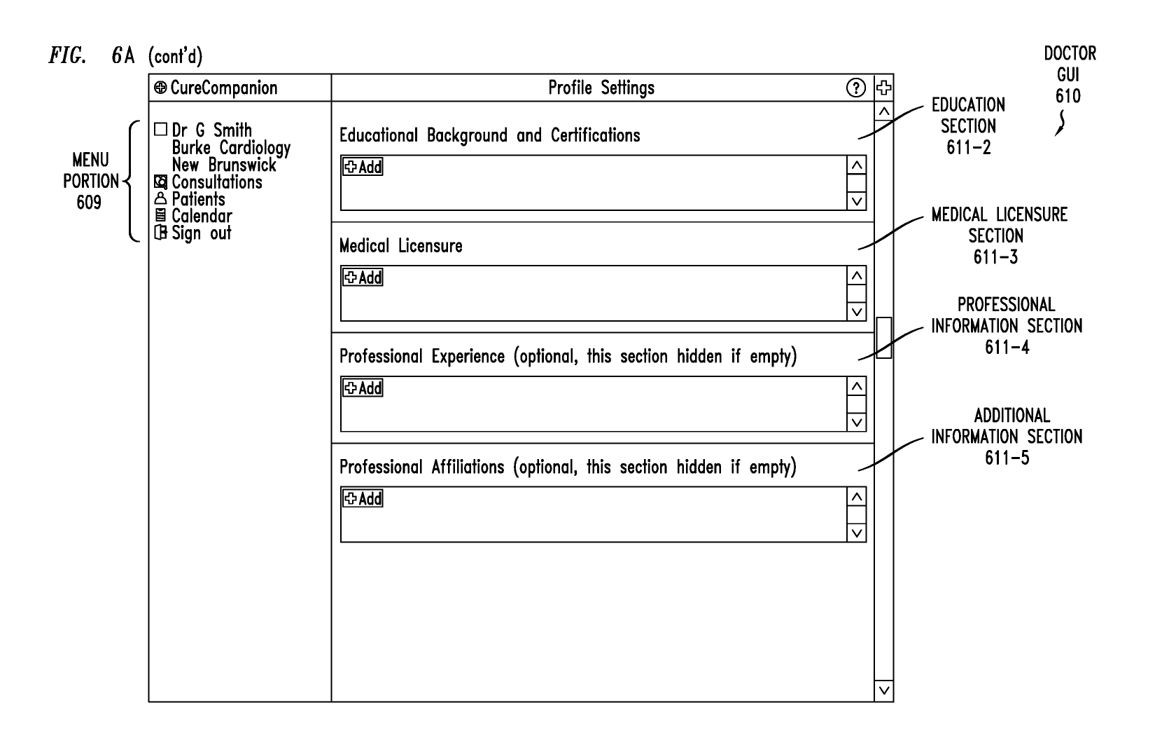

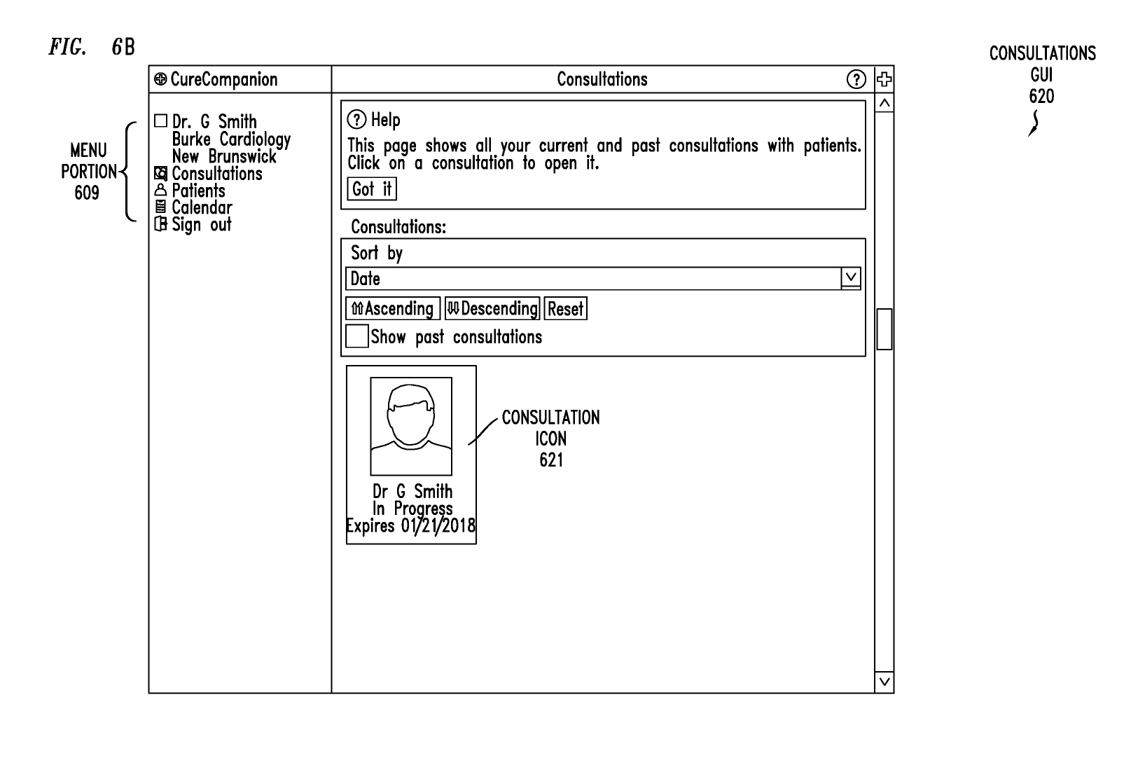

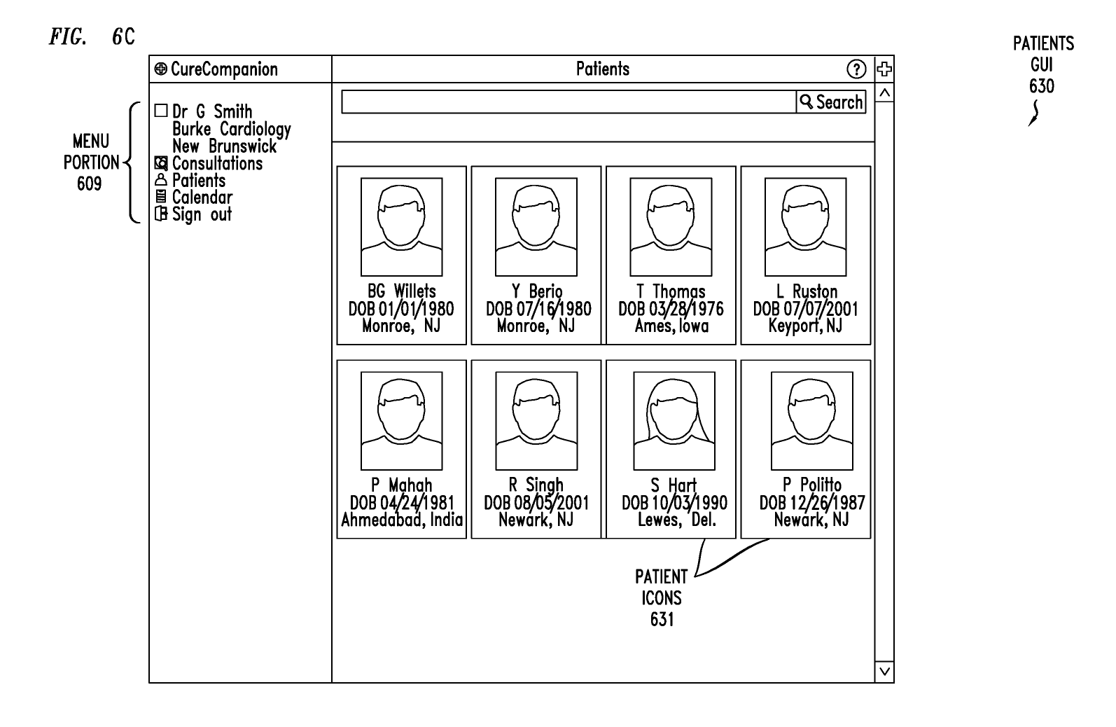

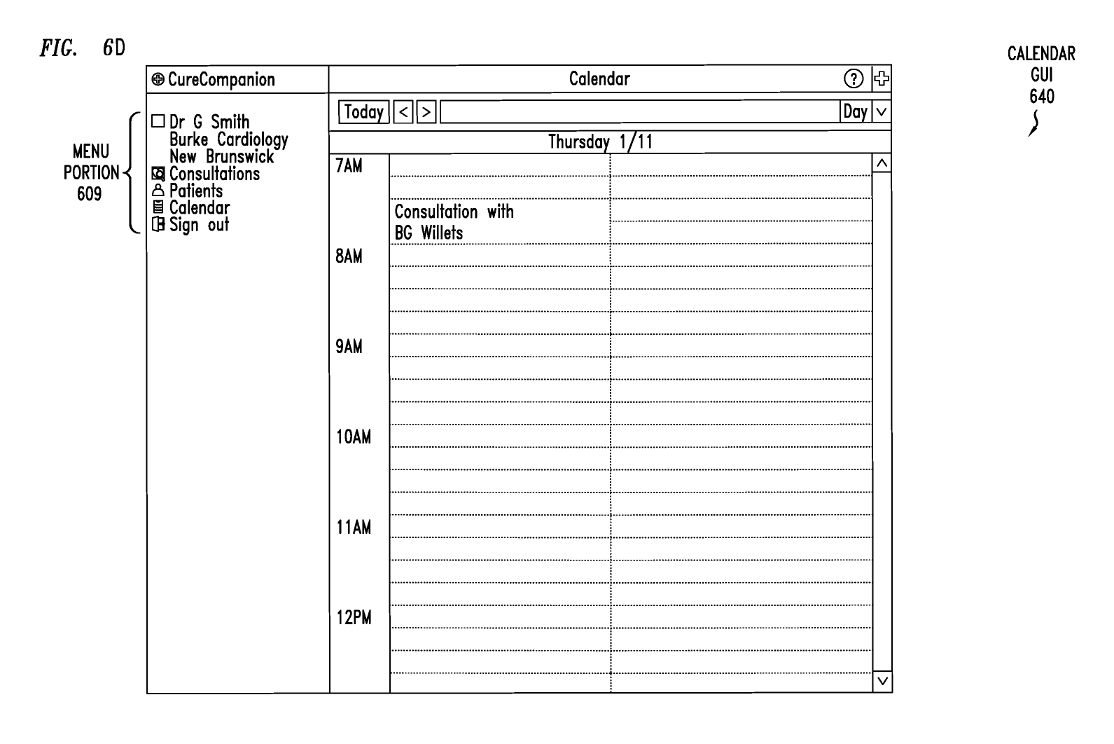

In at least some embodiments, the healthcare professional device 122 may be a communication device of the healthcare professional 121 (e.g., a smartphone, a computer, or the like). In at least some such embodiments, the healthcare professional device 122 may be running an application installed on the healthcare professional device 122 (e.g., a CureCompanion for Doctors application, as presented with respect to FIGS. 6A-6D, or other similar application).

In at least some embodiments, the healthcare professional device 122 may be a workstation. In at least some such embodiments, the workstation may be a workstation within a call center, or other similar workstation or terminal).

It will be appreciated that, although primarily presented with respect to an embodiment in which the healthcare professional 121 uses a single healthcare professional device 122 at the healthcare professional location 120 to provide healthcare services to the patient 111, the healthcare professional 121 may use multiple healthcare professional devices 122 at the healthcare professional location 120 to provide healthcare services to the patient 111 (e.g., multiple communication devices, a communication device and a workstation, or the like, as well as various combinations thereof. It will be appreciated that, although primarily presented with respect to a single healthcare professional location 120, the healthcare services system 100 may support multiple healthcare professional locations (and potentially a large number of healthcare professional locations).

The healthcare infrastructure 130 may include any healthcare infrastructure that may be used in providing healthcare services to the patient 111 at the patient location 110. For example, the healthcare infrastructure 130 may include emergency services 131 (e.g., public safety answering points (PSAPs), ambulance services, hospital emergency services, or the like, as well as various combinations thereof), healthcare services 132 (e.g., hospitals, doctors offices, urgent care facilities, in-patient facilities, out-patient facilities, or the like, as well as various combinations thereof), pharmaceutical services 133 (e.g., pharmacies or other dispensaries), or other healthcare services 134 (e.g., health data handling services, health insurance services, or the like, as well as various combinations thereof). The healthcare infrastructure 130 may include various types of healthcare resources, such as healthcare treatment facilities, healthcare transport facilities, healthcare personnel (e.g., doctors, physician assistants, nurses, or the like), communications resources, or the like, as well as various combinations thereof.

The healthcare infrastructure 130 may support treatment of the patient 111 at the patient location 110 based on various types of communications. The patient 111 at the patient location 110 may communicate with the healthcare infrastructure 130 directly (e.g., requesting emergency medical services, making appointments with other healthcare professionals, consulting other healthcare professionals, calling in prescriptions to pharmacies, or the like, as well as various combinations thereof). The healthcare profession 121 at the healthcare professional location 120 may communicate with the healthcare infrastructure 130 on behalf of the patient 111 at the patient location 110 (e.g., requesting emergency medical services for the patient 111, making appointments for the patient 111 to consult healthcare professionals, calling in prescriptions to pharmacies on behalf of the patient 111, or the like, as well as various combinations thereof). The healthcare profession 121 at the healthcare professional location 120 may communicate with the healthcare infrastructure 130 for purposes of providing healthcare services to the patient 111 (e.g., requesting emergency medical services for the patient 111, consulting with other healthcare professionals regarding the patient 111, or the like, as well as various combinations thereof).

The telemedicine server 140 may be configured to support treatment of the patient 111 at the patient location 110 by the health professional 121 at the health professional location.

The telemedicine server 140 may be configured to support use of the patient device 112 by the patient 111 at the patient location 110 (e.g., supporting use of an application installed on the patient device 112 (e.g., a patient application (e.g., a CureCompanion for Patients application), as presented with respect to FIGS. 5A-5F, or other similar application or webpage), supporting use of a telemedicine kit where the patient device 112 includes a telemedicine kit, or the like, as well as various combinations thereof). For example, where the patient 111 is using an application on the patient device 112 to obtain remote healthcare services, the telemedicine server 140 may be running a server instance of the application corresponding to the client instance of the application running on the patient device 112. The telemedicine server 140 may support various functions supported by the application running on the patient device 112, such as enabling the patient 111 to search for and find healthcare professionals, enabling the patient 111 to schedule remote appointments with healthcare professionals, enabling the patient 111 to attend remote appointments with healthcare professionals, enabling the patient 111 to pay for remote appointments with healthcare professionals, securely storing health records and history for the patient 111 across healthcare professions, or the like, as well as various combinations thereof.

The telemedicine server 140 may be configured to support use of the healthcare professional device 122 by the healthcare professional 121 at the healthcare professional location 120 (e.g., supporting use of an application installed on the healthcare professional device 122 (e.g., a healthcare professional application (e.g., a CureCompanion for Doctors application), as presented with respect to FIGS. 6A-6D, or other similar application or webpage), supporting use of a workstation where the healthcare professional device 122 includes a workstation, or the like, as well as various combinations thereof. For example, where the healthcare professional 121 is using an application on the healthcare professional device 122 to provide remote healthcare services, the telemedicine server 140 may be running a server instance of the application corresponding to the client instance of the application running on the healthcare professional device 122. The telemedicine server 140 may support various functions supported by the application running on the healthcare professional device 122, such as maintaining a public profile for enabling patients to locate the healthcare professional 121, enabling scheduling of remote appointments by patients with the healthcare professional 121, enabling patients to fill out their health history and symptoms, enabling attendance of remote appointments by patients with the healthcare professional 121 (e.g., enabling the healthcare professional 121 to review patient records, conduct a remote examination, speak with the patient, or the like), enabling the healthcare professional 121 to write provide orders for patients (e.g., test prescriptions, medicine prescriptions, doctor's notes, or the like), collecting payment for remote appointments by patients with the healthcare professional 121, or the like, as well as various combinations thereof.

The telemedicine server 140 may be composed one a set of one or more physical servers, a set of one or more virtual servers, or the like, as well as various combinations thereof. The telemedicine server 140 may be provided using a cloud-based implementation using various combinations of virtual elements. The telemedicine server 140 may be provided in various other ways.

The various communications between entities of the healthcare services system 100 may be provided in various ways. The communications may include audio communications, video communications, multimedia communications, diagnostic communications, or the like, as well as various combinations thereof. The communications between entities of the healthcare services system 100 may be secure (e.g., secure audio calls, secure video calls, or the like). The communications between entities of the healthcare services system 100 may be initiated by the patient 111, the healthcare professional 121, healthcare professionals associated with the healthcare infrastructure 130, the telemedicine server 140 (e.g., based on scheduling of communications (e.g., scheduled appointments, periodic checkups, or the like), based on monitoring of conditions associated with the patient 111 (e.g., detection of a heart condition by the telemedicine server 140 based on analysis of heart monitor data by the telemedicine server 140 or an indication of a heart condition communicated to the telemedicine server 140 by another server performing heart monitoring data analysis for the patient 111, detection of a blood sugar condition by the telemedicine server 140 based on analysis of blood sugar data by the telemedicine server 140 or an indication of a blood sugar condition communicated to the telemedicine server 140 by another server performing blood sugar monitoring data analysis for the patient 111, or the like), or the like), or the like, as well as various combinations thereof.

It will be appreciated that, although primarily presented with respect to specific numbers, types, and arrangements of locations, entities, and elements, various other numbers, types, and/or arrangements of locations, entities, and/or elements may be used to provide healthcare services system 100.

FIG. 2 depicts an example of a telemedicine bag configured for use by a patient or a healthcare provider to enable the patient to obtain remote healthcare services from a healthcare professional.

The telemedicine bag 200 of FIG. 2 is suitable for use as the patient device 112 of FIG. 1. The telemedicine bag 200 may be operated by a healthcare provider who is co-located with the patient, may be operated by the patient, may be operated by a relative or friend of the patient, or the like, as well as various combinations thereof.

The telemedicine bag 200 includes a housing 201 composed of a top portion 202 and a base portion 203. The top portion 202 and the base portion 203 are pivotally connected by a hinge 204, thereby permitting the top portion 202 to pivot with respect to the base portion 203 between a closed position and an open position. The telemedicine bag 200 is portable and, thus, the housing 201 may be considered to be a portable housing.

The telemedicine bag 200 includes a computing device 210. As illustrated in FIG. 2, the computing device 210 is disposed within the top portion 202 of the housing 201. The computing device 210 may be a touch-based computing device, such as a tablet computer, a smartphone, or the like. The computing device 210 may have various communications capabilities, including network connection capabilities for enabling the computing device 210 to communicate via communication networks (e.g., a transmitter(s), a receiver(s), network interface cards, or the like, as well as various combinations thereof), user communication capabilities for enabling the user(s) of the telemedicine bag 200 to communicate with a healthcare professional (e.g., a microphone(s), a speaker(s), a camera(s), or the like, as well as various combinations thereof), or the like, as well as various combinations thereof. The computing device 210, as discussed further below, may be configured to provide various control functions associated with use of the telemedicine bag 200 for enabling the patient to obtain remote healthcare services from a healthcare professional (e.g., collecting medical information from the patient, accessing a patient application configured for use by a patient to obtain remote healthcare services from a healthcare professional, or the like, as well as various combinations thereof). The computing device 210 may be configured to provide various control functions associated with use of the telemedicine bag 200 for enabling the patient to obtain remote healthcare services from a healthcare professional based on one or more of a patient application or webpage (e.g., which may be used by a patient located remotely from a healthcare professional to receive healthcare services from the healthcare professional, which may be used by a healthcare professional co-located with the patient to facilitate providing of healthcare services to the patient by a healthcare professional located remotely from the patient, or the like, as well as various combinations thereof), a healthcare professional application or webpage (e.g., which may be used by a healthcare professional located remotely from a patient to provide healthcare services to the patient, which may be used by a healthcare professional co-located with the patient to facilitate providing of healthcare services to the patient by a healthcare professional located remotely from the patient, or the like, as well as various combinations thereof), or the like, as well as various combinations thereof. The computing device 210 may be configured to provide various other control functions associated with use of the telemedicine bag 200 for enabling the patient to obtain remote healthcare services from a healthcare professional.

The telemedicine bag 200 includes a set of receptacles 220 configured to house a set of medical instruments 230 (illustratively, including medical instruments 230-1-230-9). As illustrated in FIG. 2, the receptacles 220 are disposed within the base portion of the housing 201. The receptacles 220 are shaped based on the medical instruments intended to be housed, respectively. The medical instruments 230 are configured to be communicatively connected to the computing device 210, thereby enabling readings of the medical instruments 230 to be obtained by the computing device 210 (e.g., for storage on the computing device 210, for storage on the telemedicine server 140 in an account of the patient, for transmission to a healthcare professional from which the patient is obtaining or will obtain remote healthcare services, or the like, as well as various combinations thereof).

The medical instruments 230 include wired medical instruments (illustratively, medical instruments 230-1-230-4, which also may be referred to more generally as wired medical instruments 230-x) that are communicatively connected to computing device 210 via wired interfaces (e.g., using Universal Serial Bus (USB) connections or other suitable wired connections). For example, one of the receptacles 220 may be configured to house a digital stethoscope 230-1, which is connected to the computing device 210 via a USB connection (although it will be appreciated that other types of connections may be used). For example, one of the receptacles 220 may be configured to house an examination camera 230-2, which is connected to the computing device 210 via a USB connection (although it will be appreciated that other types of connections may be used). For example, one of the receptacles 220 may be configured to house a digital otoscope 230-3, which is connected to the computing device 210 via a USB connection (although it will be appreciated that other types of connections may be used). For example, one of the receptacles 220 may be configured to house a digital iriscope 230-4, which is connected to the computing device 210 via a USB connection (although it will be appreciated that other types of connections may be used). It will be appreciated that fewer or more wired medical instruments may be integrated within the telemedicine bag 200.

The telemedicine bag 200 includes a wired medical instrument interface control element 240 that may be configured to control interfacing between the wired medical instruments 230-x and the computing device 210. The wired medical instrument interface control element 240 may include a set of control buttons 241-1-241-4 (collectively, control buttons 241) associated with the wired medical instruments 230-1-230-4, respectively.

The wired medical instrument interface control element 240 may be configured to, responsive to actuation of a control button 241 associated with a wired medical instrument 230-x, perform a control action associated with the wired medical instrument 230-x associated with the control button 241. The control action may include connecting the wired medical instrument 230-x to the computing device 210 via a USB connection, initializing the wired medical instrument 230-x to enter a particular state (e.g., a state in which the wired medical instrument 230-x may be configured to take a reading, a state in which the wired medical instrument 230-x may be configured to take a reading and send the reading to the computing device 210, or the like), initiating the computing device 210 to enter a particular state for the wired medical instrument 230-x (e.g., a state in which the computing device 210 may be configured to request a reading from the wired medical instrument 230-x, a state in which the computing device 210 may be configured to receive a reading from the wired medical instrument 230-x, a state in which the computing device 210 may be configured to receive a reading from the wired medical instrument 230-x and process the reading from the wired medical instrument 230-x, a state in which the computing device 210 may be configured to receive a reading from the wired medical instrument 230-x and send the reading toward the telemedicine server 140 (e.g., for storage in an account of the patient, for delivery to a healthcare professional from which the patient is obtaining or will obtain remote healthcare services, or the like, as well as various combinations thereof), or the like, as well as various combinations thereof).

The wired medical instrument interface control element 240 may be configured such that the control buttons 241 may have lighting elements associated therewith. The lighting elements of the control buttons 241 may be configured to switch between unlit and lit states depending on the status of control actions initiated when the control buttons 241 are actuated. For example, the lighting element associated with a control button 241 for a wired medical instrument 230-x may switch from an unlit state to a lit state to indicate that the wired medical instrument 230-x has been communicatively connected to the computing device 210. For example, the lighting element associated with a control button 241 for a wired medical instrument 230-x may switch from an unlit state to a lit state to indicate that the wired medical instrument 230-x has been communicatively connected to the computing device 210 and that the wired medical instrument 230-x has been initialized and is ready to take a reading, thereby informing the user of the telemedicine bag 200 that the wired medical instrument 230-x may now be used to take a reading. It will be appreciated that the lighting element associated with a control button 241 for a wired medical instrument 230-x may switch from an unlit state to a lit state in response to various other conditions. It will be appreciated that the lighting element associated with a control button 241 for a wired medical instrument 230-x also may switch from a lit state to an unlit state after the wired medical instrument 230-x is no longer needed (e.g., after a reading has been taken with the wired medical instrument 230-x, after a reading has been taken with the wired medical instrument 230-x and provided to the computing device 210, upon selection of a different control button 241 associated with a different wired medical instrument 230-x, upon selection of a wireless medical instrument, or the like).

The wired medical instrument interface control element 240 may be configured such that switching of the lighting elements associated with the control buttons 241 between unlit and lit states may be controlled by the computing device 210. The computing device 210 may be configured to control switching of the lighting elements associated with the control buttons 241 between unlit and lit states in order to guide the user of the telemedicine bag 200 through use of the wired medical instruments 230-1-230-4. The computing device 210 may be configured to control switching of the lighting elements associated with the control buttons 241 between unlit and lit states in an order determined by the computing device 210 (e.g., based on a predetermined order, based on information received from a local user of the telemedicine bag 200, based on information received from a healthcare professional from which the patient is obtaining or will obtain remote healthcare services (e.g., the nurse or doctor indicates an order in which vitals of the patient are to be taken), or the like, as well as various combinations thereof). The wired medical instrument interface control element 240 may be configured such that switching of the lighting elements associated with the control buttons 241 between unlit and lit states may be controlled by the computing device 210 in various other ways.

The wired medical instrument interface control element 240 may be configured such that the control buttons 241 may be implemented using various types of lighting elements. The lighting elements may be light emitting diodes (LEDs), lasers, or other suitable types of lighting elements.

It will be appreciated that multiple lit states (e.g., using different levels of brightness, different lighted locations, different colors, or the like) may be supported for one or more control buttons 241 in order to represent different states of the associated wired medical instruments 230-x (e.g., connected to the computing device 210, initiated for taking a reading and providing the reading to the computing device 210, reading complete and provided to the computing device 210, or the like).

It will be appreciated that, although primarily presented with respect to use of lighting elements as indicators for the control buttons 241 of the associated wired medical instruments 230-x, other suitable types of indicators may be employed to help guide the user of the telemedicine bag 200 through use of the medical instruments 230 for enabling the patient to obtain remote healthcare services.

It will be appreciated that, although primarily presented with respect to embodiments in which the wired medical instrument interface control element 240 is associated with the top portion 202 of the telemedicine bag 200, in at least some embodiments the wired medical instrument interface control element 240 may be associated with a different portion of the telemedicine bag 200 (e.g., the top portion 202 of the telemedicine bag 200, the housing 201 of the telemedicine bag 200, the computing device 210, or the like), may be provided via a display interface of the computing device 210 (e.g., using graphical versions of control buttons 241 or other suitable virtual versions of control buttons 241 which may be presented and actuated via a display interface of the computing device 210), or the like, as well as various combinations thereof.

The medical instruments 230 include wireless medical instruments (illustratively, medical instruments 230-5-230-9, which also may be referred to more generally as wireless medical instruments 230-y) that are communicatively connected to computing device 210 via wireless interfaces (e.g., using Bluetooth connections or other suitable wireless connections). For example, one of the receptacles 220 may be configured to house a blood pressure monitor 230-5, which may be connected to the computing device 210 via pairing with a Bluetooth connection (although it will be appreciated that other types of connections may be used). For example, one of the receptacles 220 may be configured to house a pulse oximeter 230-6, which may be connected to the computing device 210 via pairing with a Bluetooth connection (although it will be appreciated that other types of connections may be used). For example, one of the receptacles 220 may be configured to house a glucometer 230-7, which may be connected to the computing device 210 via pairing with a Bluetooth connection (although it will be appreciated that other types of connections may be used). For example, one of the receptacles 220 may be configured to house a spirometer 230-8, which may be connected to the computing device 210 via pairing with a Bluetooth connection (although it will be appreciated that other types of connections may be used). For example, one of the receptacles 220 may be configured to house a thermometer 230-9, which may be connected to the computing device 210 via pairing with a Bluetooth connection (although it will be appreciated that other types of connections may be used). It will be appreciated that fewer or more wireless medical instruments may be integrated within the telemedicine bag 200.

The telemedicine bag 200 includes a wireless medical instrument interface control capability that is configured to control interfacing between the wireless medical instruments and the computing device 210. The wireless medical instrument interface control capability may be provided by the computing device 210, such as by enabling selection of the wireless medical instruments using touch-screen capabilities of the computing device 210 (e.g., the wireless medical instruments may be paired with the computing device 210 via Bluetooth responsive to actuation of buttons on the touchscreen display of computing device 210). It will be appreciated that interfacing between the wireless medical instruments and the computing device 210 may be provided in other ways.

The wireless medical instrument interface control capability may be configured to, responsive to actuation of a control button associated with a wireless medical instrument 230-y (e.g., a graphical element on the touch-screen display of the computing device 210), perform a control action associated with the wireless medical instrument 230-y associated with the control button associated with a wireless medical instrument 230-y. The control action may include connecting the wireless medical instrument 230-y to the computing device 210 via a wireless connection (e.g., pairing via Bluetooth), initializing the wireless medical instrument 230-y to enter a particular state (e.g., a state in which the wireless medical instrument 230-y may be configured to take a reading, a state in which the wireless medical instrument 230-y may be configured to take a reading and send the reading to the computing device 210, or the like), initiating the computing device 210 to enter a particular state for the wireless medical instrument 230-y (e.g., a state in which the computing device 210 may be configured to request a reading from the wireless medical instrument 230-y, a state in which the computing device 210 may be configured to receive a reading from the wireless medical instrument 230-y, a state in which the computing device 210 may be configured to receive a reading from the wireless medical instrument 230-y and process the reading from the wireless medical instrument 230-y, a state in which the computing device 210 may be configured to receive a reading from the wireless medical instrument 230-y and send the reading toward the telemedicine server 140 (e.g., for storage in an account of the patient, for delivery to a healthcare professional from which the patient is obtaining or will obtain remote healthcare services, or the like, as well as various combinations thereof), or the like, as well as various combinations thereof).

The wireless medical instrument interface control capability may be configured such that the control buttons may be presented and controlled in various ways. The control buttons for the wireless medical instruments 230-y may be presented via the touch-screen display of the computing device 210 in various ways, such as where the control buttons for the wireless medical instruments 230-y may all be presented via the touch-screen display of the computing device 210 (e.g., such that any of the wireless medical instruments 230-y may be used at any time), may be presented via the touch-screen display of the computing device 210 individually in a serial manner in order to guide the healthcare professional from which the patient is obtaining or will obtain remote healthcare services through use of the wireless medical instruments 230-y, or the like, as well as various combinations thereof. The control buttons for the wireless medical instruments 230-y may be controlled to provide status information regarding availability and use of the wireless medical instruments 230-y. The control buttons for the wireless medical instruments 230-y may be controlled to switch between states (e.g., selectable versus un-selectable, highlighted versus un-highlighted, enlarged versus not enlarged, flashing versus not flashing, or the like, as well as various combinations thereof) depending on the status of control actions initiated when the control buttons 241 are actuated. For example, the control button associated with a wireless medical instrument 230-y may switch states to indicate that the wireless medical instrument 230-y has been communicatively connected to the computing device 210. For example, the control button associated with a wireless medical instrument 230-y may switch states to indicate that the wireless medical instrument 230-y has been communicatively connected to the computing device 210 and that the wireless medical instrument 230-y has been initialized and is ready to take a reading, thereby informing the user of the telemedicine bag 200 that the wireless medical instrument 230-y may now be used to take a reading. It will be appreciated that the control button associated with a wireless medical instrument 230-y may switch states in response to various other conditions. It will be appreciated that the control button associated with a wireless medical instrument 230-y may switch states in response to an indication that the wireless medical instrument 230-y is no longer needed (e.g., after a reading has been taken with the wireless medical instrument 230-y, after a reading has been taken with the wireless medical instrument 230-y and provided to the computing device 210, upon selection of a different control button associated with a different wireless medical instrument 230-y, upon selection of a wired medical instrument, or the like).

The wireless medical instrument interface control capability may be configured such that switching of the control buttons between states may be controlled by the computing device 210. The computing device 210 may be configured to control switching of the control buttons between states in order to guide the user of the telemedicine bag 200 through use of the wireless medical instruments 230-y. The computing device 210 may be configured to control switching of the control buttons between states in an order determined by the computing device 210 (e.g., based on a predetermined order, based on information received from a local user of the telemedicine bag 200, based on information received from a healthcare professional from which the patient is obtaining or will obtain remote healthcare services (e.g., the nurse or doctor indicates an order in which vitals of the patient are to be taken), or the like, as well as various combinations thereof). The wireless medical instrument interface control capability may be configured such that switching of the control buttons between states may be controlled by the computing device 210 in various other ways.

The wireless medical instrument interface control capability may be configured such that the control buttons may be presented on the computing device 210 in various ways (e.g., using various types of graphical objects, arranged in various ways with respect to each other or other graphical elements, or the like, as well as various combinations thereof).

It will be appreciated that multiple display states (e.g., using different colors, different sizes, different locations, different status indicators, or the like) may be supported for one or more control buttons in order to represent different states of the associated wireless medical instruments 230-y (e.g., connected to the computing device 210, initiated for taking a reading and providing the reading to the computing device 210, reading complete and provided to the computing device 210, or the like).

It will be appreciated that, although primarily presented with respect to use of graphical elements as the control buttons of the associated wireless medical instruments 230-y, other suitable types of elements may be employed to help guide the user of the telemedicine bag 200 through use of the medical instruments 230 for enabling the patient to obtain remote healthcare services.

It will be appreciated that, although primarily presented with respect to use of the control buttons 241 as control buttons for the wired medical instruments 230-x and use of graphical elements as the control buttons of the associated wireless medical instruments 230-y, control buttons for the wired medical instruments 230-x may be provided using various other types of elements (e.g., using graphical elements of the touch-screen display of computing device 210 similar to those used for the wireless medical instruments 230-y), control buttons for the wireless medical instruments 230-y may be provided using various other types of elements (e.g., using physical control buttons similar to the control buttons used for the wired medical instruments 230-x), or the like, as well as various combinations thereof.

It will be appreciated that, although the telemedicine bag 200 is primarily presented herein as having specific types and arrangements of elements (e.g., computing device 210, medical instruments 230, and the like), the telemedicine bag 200 may include fewer or more elements (e.g., additional computing devices 210, fewer or more receptacles 220, fewer or more medical instruments 230, or the like), may support other arrangements of elements (e.g., other arrangements of the computing device 210 with respect to the medical instruments 230, other arrangements of the receptacles 220 with respect to each other, other arrangements of the receptacles 220 and the medical instruments 230 with respect to each other, or the like), or the like, as well as various combinations thereof. FIG. 3 depicts an example of a telemedicine cart configured for use by a patient or a healthcare provider to enable the patient to obtain remote healthcare services from a healthcare professional.

The telemedicine cart 300 of FIG. 3 is suitable for use as the patient device 112 of FIG. 1. The telemedicine cart 300 of FIG. 3 may be considered to be a modified version of the telemedicine bag 200 presented with respect to FIG. 2. The telemedicine cart 300 may be operated by a healthcare provider who is co-located with the patient or may be operated by the patient.

The telemedicine cart 300 of FIG. 3 is similar to the telemedicine bag 200 of FIG. 2 in certain ways (e.g., in terms of the types of devices included and the manner in which those devices operate), but is different from the telemedicine bag 200 of FIG. 2 in certain ways (e.g., in terms of the arrangement of the devices).

The telemedicine cart 300 includes a base portion 301, a stand portion 302, and a medical examination portion 303.

The base portion 301 provides a base for the stand portion 302 and, thus, for the medical examination portion 303. The base portion 301 includes four wheels, which enable the telemedicine cart 300 to be moved relatively easily (e.g., between rooms, between buildings, or the like). The base portion 301 may be configured to have a relatively wide base (e.g., relative to the height and weight of the stand portion 302 and the medical examination portion 303) so as to eliminate or reduce the potential for the telemedicine cart 300 to fall over. It will be appreciated that the base portion 301 may be arranged in other ways, may include other elements or support other features or functions, or the like.

The stand portion 302 connects the medical examination portion 303 to the base portion 301. The stand portion 302 may be any height conducive to use of the medical examination portion 303 by a patient or a healthcare professional. The stand portion 302 may be configured to support adjustability of the height of the medical examination portion 303 to facilitate use of the medical examination portion 303 by a patient or a healthcare professional. It will be appreciated that the stand portion 302 may be arranged in other ways, may include other elements or support other features or functions, or the like.

The medical examination portion 303, as illustrated in FIG. 3, may include the same or substantially the same elements as the telemedicine bag 200 of FIG. 2; however, as illustrated in FIG. 3, the arrangement of the elements is different than the telemedicine bag 200 of FIG. 2. It also will be appreciated that the set of elements of medical examination portion 303 may be different than the set of elements of the telemedicine bag 200 of FIG. 2.

The medical examination portion 303 of the telemedicine cart 300 includes a computing device 310, a set of medical instrument support elements 320, and a set of medical instruments 330.

The computing device 310 includes a display portion 311 and a control portion 312. The display portion 311 may be a tablet or other computing device, a television or other display device, or the like. The control portion 312 may be a mouse, a mouse and a keyboard, or the like. The computing device 310 also may include a pan-tilt-zoom (PTZ) camera 313. The computing device 310 may be configured to operate in a manner similar to the computing device 210 of the telemedicine bag 200 of FIG. 2. It will be appreciated that, although omitted from FIG. 3 for purposes of clarity, the computing device 310 may be a computing device in which the display portion 311 and the control portion 312 are at least partially integrated (e.g., a laptop computer, a tablet computer, or the like).

The set of medical instrument support elements 320 includes a shelf 321 and a drawer 322. The shelf 321 supports placement of medical instruments 330 before, during, and after use of the medical instruments 330. The drawer 322 supports storage of medical instruments 330 when the medical instruments 330 are not being used. It will be appreciated that the medical instrument support elements 320 may include fewer or more shelves, fewer or more drawers, one or more other types of medical instrument support elements, or the like, as well as various combinations thereof.

The medical instruments 330 include wired medical instruments (illustratively, medical instruments 330-1-330-4) that are communicatively connected to computing device 310 via wired interfaces (e.g., using Universal Serial Bus (USB) connections or other suitable wired connections). For example, the wired medical instruments 330-x include a digital stethoscope 330-1, an examination camera 330-2, a digital otoscope 330-3, and a digital iriscope 330-4. The wired medical instruments 330-x may be housed within a particular compartment(s) of the drawer 322 (e.g., based on their wired connectivity to the computing device 310).

The medical instruments 330 include wireless medical instruments (illustratively, medical instruments 330-5-330-9) that are communicatively connected to computing device 310 via wireless interfaces (e.g., using Bluetooth connections or other suitable wireless connections). For example, the wireless medical instruments 330-y include a blood pressure monitor 330-5, a pulse oximeter 330-6, a glucometer 330-7, a spirometer 330-8, and a thermometer 330-9.

It will be appreciated that fewer or more wireless medical instruments may be integrated within the telemedicine cart 300.

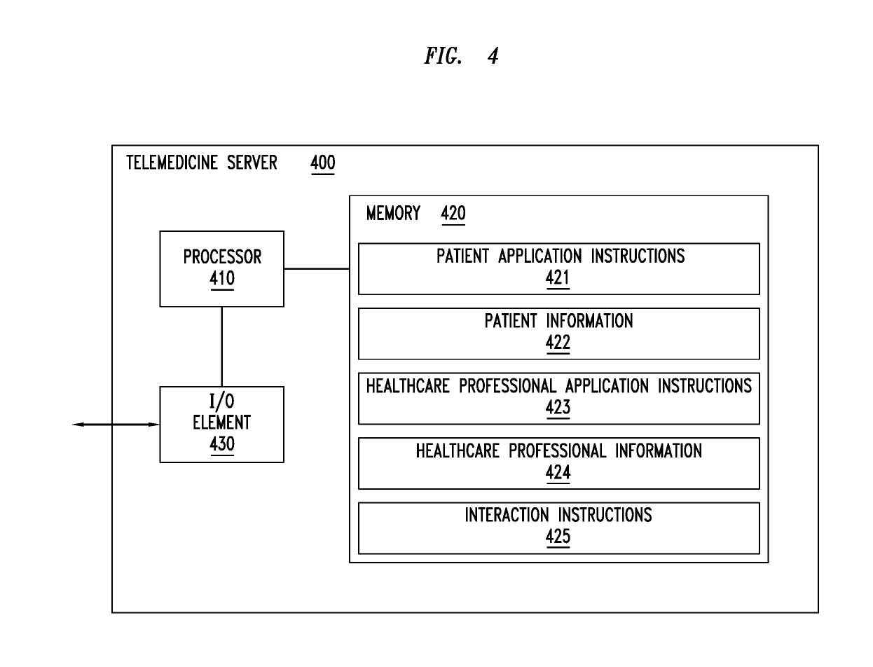

FIG. 4 depicts an example of a telemedicine server configured to support a telemedicine platform configured to enable patients to obtain remote healthcare services from healthcare professionals.

The telemedicine server 400 of FIG. 4 is suitable for use as the telemedicine server 140 of FIG. 1.

The telemedicine server 400 may be configured to support a telemedicine platform configured to support integration of the applications (e.g., the patient application and the healthcare professional application) and telemedicine devices in order to support various remote healthcare scenarios.

The telemedicine server 400 includes a processor 410, a memory 420, and an input-output (I/O) element 430.

The processor 410 is communicatively connected to the memory 420 and the I/O element 430. The processor 410 may be configured to perform processing in order to provide various functions of the telemedicine server 400 (e.g., retrieving instructions from the memory 420 and executing the instructions, retrieving information from memory 420 and processing the information, storing information in memory 420, controlling communications with various other devices (e.g., patient devices, healthcare professional devices, telemedicine devices, or the like, as well as various combinations thereof), or the like, as well as various combinations thereof).

The memory 420 may be configured to store various instructions and information which may be used by processor 410 to provide various functions of the telemedicine server 400. For example, the memory 420 may store patient-related instructions and information.

For example, the memory 420 may store patient application instructions 421 related to support of the patient application used by patients (e.g., the patient application presented with respect to FIGS. 5A-5F). For example, the memory 420 may store patient information 422 for patients (e.g., personal information, medical history, medical consultation information, or the like, as well as various combinations thereof), which may be obtained from various sources (e.g., via the patient application, via the healthcare professional application, or the like, as well as various combinations thereof). It will be appreciated that, although presented as being stored in memory 420, the patient information 422 for patients may be stored in other devices (e.g., other storage devices, databases such as telemedicine database 141 of FIG. 1, or the like, as well as various combinations thereof). The memory 420 may store various other patient-related instructions and information.

For example, the memory 420 may store healthcare professional related instructions and information. For example, the memory 420 may store healthcare processional application instructions 423 related to support of the patient application used by patients (e.g., the healthcare professional application presented with respect to FIGS. 6A-6D). For example, the memory 420 may store healthcare professional information 424 for healthcare professionals (e.g., personal information, medical credentials, consultation availability, or the like, as well as various combinations thereof), which may be obtained from various sources (e.g., via the healthcare processional application, via the patient application, or the like, as well as various combinations thereof). It will be appreciated that, although presented as being stored in memory 420, the healthcare professional information 424 for patients may be stored in other devices (e.g., other storage devices, databases such as telemedicine database 141 of FIG. 1, or the like, as well as various combinations thereof). The memory 420 may store various other healthcare professional related instructions and information.

For example, the memory 420 may store interaction instructions 425, which may include one or more of instructions related to interaction between the patient application and the healthcare professional application (e.g., for scheduling consultations, conducting consultations, or the like), instructions related to interaction between applications (e.g., the patient application and/or the healthcare professional application) and telemedicine devices (e.g., for guiding medical exams, for collecting information obtained during medical exams, for distributing information collected during medical exams, or the like), or the like, as well as various combinations thereof.

The memory 420 may store various other types of instructions and information related to performing various functions presented herein.

The I/O element 430 may be configured to support communications of the telemedicine server 400. The communications may include various messages, requests, commands, information, or the like, as well as various combinations thereof. The communications of the telemedicine server 400 may include communications related to use of patient applications by patients, communications related to use of healthcare provider applications, communications related to collection and distribution of medical information based on use of telemedicine devices, or the like, as well as various combinations thereof.

The telemedicine server 400 may include various other elements configured to support various functions of the telemedicine server 400.

FIGS. 5A-5F depict examples of graphical user interfaces of a patient application configured for use by a patient to obtain remote healthcare services from a healthcare professional.

The patient application supporting the GUIs of FIGS. 5A-5F may be running on the patient device 112 of FIG. 1.

The patient application supporting the GUIs of FIGS. 5A-5F is a user-friendly application configured to enable patients to manage their healthcare (e.g., managing their health records, consulting doctors remotely, or the like, as well as various combinations thereof). For example, the patient application supporting the GUIs of FIGS. 5A-5F may be configured to enable a patient to securely store health records and history, find a doctor and schedule a video appointment, pay the doctor using a credit card or other form of payment, consult the doctor via a secure video call, and so forth. The various functions supported by the patient application may be further understood by considering the GUIs of FIGS. 5A-5F.

The GUIs of the patient application become accessible to the user when the user accesses the patient application.

The GUIs of the patient application include a PATIENT GUI 510 (presented in FIG. 5A), a CONSULTATIONS GUI 520 (presented in FIG. 5B), a DOCTORS GUI 530 (presented in FIG. 5C), a PAST MEDICAL HISTORY GUI 540 (presented in FIG. 5D), a VITALS AND DIAGNOSTICS GUI 550 (presented in FIG. 5E), and a NEW CONSULTATION REQUEST GUI 560 (presented in FIG. 5F).

The GUIs of the patient application each include a menu portion 509 by which the user of the patient application may navigate between GUIs of the patient application. The menu portion 509 includes a PATIENT button 501 for navigation to the PATIENT GUI 510, a CONSULTATIONS button 502 for navigation to the CONSULTATIONS GUI 520, a DOCTORS button 503 for navigation to the DOCTORS GUI 530, a PAST MEDICAL HISTORY button 504 for navigation to the PAST MEDICAL HISTORY GUI 540, and a VITALS AND DIAGNOSTICS button 505 for navigation to the VITALS AND DIAGNOSTICS GUI 550. It is noted that the user may navigate to the NEW CONSULTATION REQUEST GUI 560 from the DOCTORS GUI 530 (e.g., after selection of a doctor so that the patient may request a consultation with the selected doctor). The menu portion 509 also includes a SIGN OUT button 506, by which the user may sign out of the patient application. It will be appreciated that the menu portion 509 may be arranged in other ways (e.g., a different ordering of the buttons), the menu portion 509 may include fewer or more buttons (as well as different buttons), or the like, as well as various combinations thereof.

The PATIENT GUI 510 may be configured to support entry and presentation of personal information of the patient. The PATIENT GUI 510 includes a profile picture 511. The PATIENT GUI 510 includes various patient information fields 512 in which personal information of the patient may be presented and in which personal information of the patient may be added or modified. For example, the PATIENT GUI 510 may include various patient information fields 512 storing patient information such as patient username and password information for the patient application, patient name, patient date of birth, patient gender, patient address, patient contact information (e.g., phone number(s), email, or the like), preferred pharmacy, insurance information, or the like, as well as various combinations thereof. The PATIENT GUI 510 includes a button 513 configured to enable the patient to save information entered into the patient information fields 512. The PATIENT GUI 510 includes a button 514 configured to enable the patient to add family members to his or her patient account. It will be appreciated that the PATIENT GUI 510 may be arranged in other ways, may include various other types of information or controls, or the like, as well as various combinations thereof.

The CONSULTATIONS GUI 520 may be configured to support consultations by the patient with doctors, enabling the patient to request new consultations and review previous consultations. The CONSULTATIONS GUI 520 presents each previous consultation of the patient, individually, using consultation icons 521. The consultation icon 521 for a previous consultation includes a photo of the doctor consulted during that consultation, the name of the doctor consulted during that consultation, a consultation status indicator (e.g., COMPLETED to indicate that the consultation was completed or OPEN to indicate that the consultation has not yet been completed), and a date that the consultation was started. It will be appreciated that the consultation icons 521 may present less or more information (including different information). The consultation icons 521 are selectable to access a CONSULTATION DETAILS GUI (omitted for purposes of clarity). The CONSULTATION DETAILS GUI may include information such as the date and time of the consultation, doctor details of the doctor consulted, symptoms identified and discussed during the consultation, report orders, prescriptions, doctor's notes, or the like, as well as various combinations thereof. The CONSULTATION DETAILS GUI also may include various control elements, such as control elements by which the patient may purchase a new consultation with the doctor, initiate an audio or video call with the doctor, or the like, as well as various combinations thereof. The CONSULTATIONS GUI 520 includes sort controls 522 by which the patient may sort the previous consultations and, thus, control the manner in which the consultation icons 521 are presented to the patient (e.g., sorting by date in ascending or descending order, sorting by doctor name in ascending or descending order, or the like). The CONSULTATIONS GUI 520 includes a new consultation request button 523 by which the patient may request a consultation with a doctor. The selection of the new consultation request button 523 results in display of the DOCTORS GUI 530. It will be appreciated that the CONSULTATIONS GUI 520 may be arranged in other ways, may include various other types of information or controls, or the like, as well as various combinations thereof.

The DOCTORS GUI 530 may be configured to support consultations by the patient with doctors, enabling the patient to request new consultations. The DOCTORS GUI 530 may initially present preferred doctors of the patient (as illustrated in FIG. 5C) or may not initially present any doctors until the patient request a list of potential doctors (omitted for purposes of clarity). The DOCTORS GUI 530 includes a set of doctor filter controls 531 by which the patient may specify parameters to be considered in determining a list of potential doctors for consultation by the patient. The doctor filter controls 531 may include various drop down menu, radio buttons, or the like. The doctor filter controls 531 may enable the patient to request potential doctors based on criteria such as area of specialty (e.g., options may include all specialties, family medicine, internal medicine, cardiology, oncology, and so forth), location (e.g., based on country, state, county, city, zip code, or the like), availability (e.g., whether or not the doctor is accepting new patients, whether or not the doctor is currently accepting consultation requests, or the like), including or excluding doctors from other locations (e.g., other cities, other states, other countries, or the like), or the like, as well as various combinations thereof. The DOCTORS GUI 530, responsive to a request from the patient and based on the settings in the doctor filter controls 531, presents each potential doctor which may be selected by the patient for consultation. The DOCTORS GUI 530 presents each potential doctor, individually, using doctor icons 532. The doctor icon 521 for a potential doctor includes a photo of the doctor, an office address of the doctor, a hospital affiliation of the doctor, a specialty area of the doctor, or the like, as well as various combinations thereof. It will be appreciated that the doctor icons 532 may present less or more information (including different information). The doctor icons 532 are selectable to access a DOCTOR DETAILS GUI (omitted for purposes of clarity). The DOCTOR DETAILS GUI may include information such as the consultation fee charged by the doctor, a background description of the doctor, associated healthcare facilities of the doctor, educational background and certifications of the doctor, medical licensure information of the doctor, or the like, as well as various combinations thereof. The DOCTOR DETAILS GUI also may include various control elements, such as control elements by which the patient may save the doctor profile for later use, purchase a new consultation with the doctor, or the like, as well as various combinations thereof. The DOCTOR DETAILS GUI may include a CONSULT control element which, when selected, results in display of the NEW CONSULTATION REQUEST GUI of FIG. 5F (discussed further below). It will be appreciated that the DOCTORS GUI 530 may be arranged in other ways, may include various other types of information or controls, or the like, as well as various combinations thereof.

The PAST MEDICAL HISTORY GUI 540 may be configured to support entry and presentation of past medical history of the patient. The PAST MEDICAL HISTORY GUI 540 includes various past medical history sections 541 in which past medical history information of the patient may be presented and in which past medical history information of the patient may be added or modified. For example, the PAST MEDICAL HISTORY GUI 540 includes a medications section 541-1 in which medications of the patient may be entered and presented. The medications section 541-1 may include a control element configured to support addition of medications to the medications section 541-1 (e.g., selection of the control element, such as a button or other control element, results in presentation of a window or GUI via which the patient may enter medication information for the medication, such as name, strength, directions, prescriber, date started, date ended, or the like, as well as various combinations thereof). The medications section 541-1 may include respective medication elements for the respective medications added to the medications section 541-1, each of which may include information associated with prescription of the respective mediation to the patient (e.g., name, strength, directions, prescriber, date started, date ended, or the like, as well as various combinations thereof) and each of which may have a control element which, when selected, enables updates to or deletion of the respective medication elements. For example, the PAST MEDICAL HISTORY GUI 540 includes an allergies section 541-2 in which allergies of the patient may be entered and presented. The allergies section 541-2 may include a control element configured to support addition of allergies to the allergies section 541-2 (e.g., selection of the control element, such as a button or other control element, results in presentation of a window or GUI via which the patient may enter allergy information for the allergy, such as allergy type, reaction that results, date that allergy first started, or the like, as well as various combinations thereof). The allergies section 541-2 may include respective allergy elements for the respective allergies added to the allergies section 541-2, each of which may include information associated with the patient allergy (e.g., allergy type, reaction that results, date that allergy first started, or the like, as well as various combinations thereof) and each of which may have a control element which, when selected, enables updates to or deletion of the respective allergies. For example, the PAST MEDICAL HISTORY GUI 540 includes a medical conditions section 541-3 in which medical conditions of the patient may be entered and presented. The medical conditions section 541-3 may include a control element configured to support addition of medical conditions to the medical conditions section 541-3 (e.g., selection of the control element, such as a button or other control element, results in presentation of a window or GUI via which the patient may enter medical condition information for the medical condition, such as condition name, date that condition was first diagnosed, details regarding the condition, or the like, as well as various combinations thereof). The medical conditions section 541-3 may include respective medical condition elements for the respective medical conditions added to the medical conditions section 541-3, each of which may include information associated with the medical condition of the patient (e.g., condition name, date that condition was first diagnosed, details regarding the condition, or the like, as well as various combinations thereof) and each of which may have a control element which, when selected, enables updates to or deletion of the respective medical conditions. For example, the PAST MEDICAL HISTORY GUI 540 includes a surgeries section 541-4 in which surgeries undergone by the patient may be entered and presented. The surgeries section 541-4 may include a control element configured to support addition of surgeries to the surgeries section 541-4 (e.g., selection of the control element, such as a button or other control element, results in presentation of a window or GUI via which the patient may enter surgery information for the surgery, such as surgery type, date, location, doctor comments, or the like, as well as various combinations thereof). The surgeries section 541-4 may include respective surgery elements for the respective surgeries added to the surgeries section 541-4, each of which may include information associated with the surgery undergone by the patient (e.g., surgery type, date, location, doctor comments, or the like, as well as various combinations thereof) and each of which may have a control element which, when selected, enables updates to or deletion of the respective surgery. For example, the PAST MEDICAL HISTORY GUI 540 includes a family history section 541-5 in which family history of the patient may be entered and presented. The family history section 541-5 may include a control element configured to support addition of family history information to the family history section 541-5 (e.g., selection of the control element, such as a button or other control element, results in presentation of a window or GUI via which the patient may enter family history information for a family member, such as name, relationship to the patient, details regarding medical history relevant to the patient, or the like, as well as various combinations thereof). The family history section 541-5 may include respective family history elements for respective family members added to the family history section 541-5, each of which may include information associated with the family member (e.g., name, relationship to the patient, details regarding medical history relevant to the patient, or the like, as well as various combinations thereof) and each of which may have a control element which, when selected, enables updates to or deletion of the respective family member and associated family history information for the respective family member. The social history section 541-6 may include a set of questions adapted to collect social history information of the patient. The social history section 541-6 may include respective social history question elements for respective questions which may be asked to obtain social history information from the patient, each of which may include an indication of the question and a set of answers selectable by the patient to answer the respective question (e.g., questions pertaining to smoking, alcohol consumption, drug use, eating habits, exercise habits, or the like, as well as various combinations thereof). It will be appreciated that the PAST MEDICAL HISTORY GUI 540 may be arranged in other ways, may include various other types of information or controls, or the like, as well as various combinations thereof.

The VITALS AND DIAGNOSTICS GUI 550 may be configured to support collection and presentation of various types of medical information of the patient. The VITALS AND DIAGNOSTICS GUI 550 includes a button 551 configured to enable the patient to initiate collection of medical information of the patient. The collection of the medical information of the patient may be in the form of entry of the information by the user or collection of the information from a medical instrument (e.g., a medical instrument of a patient device 112, such as a medical instrument 230 of the telemedicine bag 200 of FIG. 2, a medical instrument of 330 of the telemedicine cart 300 of FIG. 3, or the like). The selection of the button 551 may result in display of a mechanism (e.g., a popup window, a separate GUI, or the like) by which the user may select the type of medical information (e.g., diagnostic type to be entered (e.g., height, weight, temperature, blood pressure, or the like, as well as various combinations thereof) or collected (e.g., a reading from a digital stethoscope, a picture or video from an examination camera, a reading from a digital otoscope, a reading from a digital iriscope, a reading from a blood pressure monitor, a reading from a pulse oximeter, a reading from a glucometer, a reading from a spirometer, a reading from a thermometer, a magnetic resonance image (MRI), a computed tomography (CT) scan, an electroencephalogram (EEG), an electrocardiogram (EKG), or the like, as well as various combinations thereof). The selection by the user of the type of medical information to be collected may then result in initiation of an action, which may vary based on whether the medical information is to be entered by the user or collected. For example, where the medical information is to be entered by the user, the selection by the user of the type of medical information to be collected may result in presentation of a mechanism (e.g., a popup window, a separate GUI, or the like) by which the user may enter the information. For example, where the medical information is to be collected from a medical instrument, the selection by the user of the type of medical information to be collected may result in initiation of a request or a command to a user (e.g., to the patient or another user of a patient device that has the corresponding medical instrument associated therewith) or to the patient device that has the corresponding medical instrument associated therewith. It will be appreciated that collection of medical information may include a combination of entry of medical information by a user and collection of medical information from one or more medical instruments. The VITALS AND

DIAGNOSTICS GUI 550 presents each previous medical information collection event, individually, using medical information icons 552. The medical information icon 552 for a previous medical information collection event includes a title or description of the medical information collection event (e.g., vital signs, spirometry, x-ray, CBC report, and so forth) and the details of the medical information collection event which, it will be appreciated, may vary for different types of medical information collection events. For example, for a vital signs collection event, the medical information may include the date and time at which the vital signs were taken, the height and weight of the patient, the temperature and blood pressure of the patient, and the pule-ox, heart rate, and respiratory rate of the patient. For example, for a spirometry collection event, the medical information may include the date and time at which the measurements were taken and the various spirometry measures taken (e.g., PEF, FEV1, FEV6, FEV1/FEV6, FEF, or the like). It will be understood that other medical information may be collected for other types of medical information collection events. It will be appreciated that the medical information icon 552 may present less or more information (including different information) for given medical information collection event types. The medical information icon 552 for a previous medical information collection event may be selectable in order to access a MEDICAL INFORMATION DETAILS GUI (omitted for purposes of clarity). The MEDICAL INFORMATION DETAILS GUI may include additional information for the associated medical information collection event. It will be appreciated that the VITALS AND DIAGNOSTICS GUI 550 may be arranged in other ways, may include various other types of information or controls, or the like, as well as various combinations thereof.

The NEW CONSULTATION REQUEST GUI 560 may be configured to support generation and submission of a new consultation request by the patient. The NEW CONSULTATION REQUEST GUI 560 includes various new consultation request sections 561 which form part of the new consultation request by the patient for a consultation with a doctor selected by the patient. For example, the NEW CONSULTATION REQUEST GUI 560 includes a doctor information section 561-1 in which information about the doctor is displayed (e.g., the profile picture, location, practice area, or the like). For example, the NEW CONSULTATION REQUEST GUI 560 includes an appointment information section 561-2 which provides an indication of the fee charged by the doctor for a remote consultation, an indication of the type of consultation (e.g., a secure video call with 3 days of messaging), and a capability by which the patient may select a date and time for the consultation (e.g., a SELECT DATE button which, when selected, opens a calendar window in which the patient may select the date and time for the consultation). For example, the NEW CONSULTATION REQUEST GUI 560 includes a symptoms section 561-3 in which symptoms of the patient may be entered and presented. The symptoms section 561-3 may include a control element configured to support addition of symptoms to the symptoms section 561-3 (e.g., selection of the control element, such as a button or other control element, results in presentation of a window or GUI via which the patient may enter symptom information for the symptom, such as symptom name, symptom severity, the date and time that the symptom started, the date and time that the symptom stopped, additional symptom description information, or the like, as well as various combinations thereof). The symptoms section 561-3 may include respective symptom elements for the respective symptoms added to the symptoms section 561-3, each of which may include information associated with that symptom of the patient (e.g., symptom name, symptom severity, the date and time that the symptom started, the date and time that the symptom stopped, additional symptom description information, or the like, as well as various combinations thereof) and each of which may have a control element which, when selected, enables updates to or deletion of the respective symptom. For example, the NEW CONSULTATION REQUEST GUI 560 includes a past medical history section 561-4 in which past medical history information of the patient may be entered and presented. The past medical history information that is presented in the past medical history section 561-4, where it already exists, may be pulled in automatically for the patient based on previous entry of the past medical history information via the PAST MEDICAL HISTORY GUI 540. The past medical history information that is presented in the past medical history section 561-4, where it does not already exist, may be entered by the patient in a manner similar to that described with respect entry of past medical history information via the PAST MEDICAL HISTORY GUI 540. The past medical history information that is presented in the past medical history section 561-4, where it already exists, may be modified or deleted by the patient. For example, the NEW CONSULTATION REQUEST GUI 560 includes a message section 561-5 in which the patient may enter a message for the doctor. The message section 561-5 may be provided in the form of a dialog box or other suitable graphical element. The patient may use message section 561-5 to enter any other information that the patient would like to provide to the doctor in advance of the consultation. For example, the NEW CONSULTATION REQUEST GUI 560 includes a payment section 561-6 in which the patient may enter payment information in order to pay the doctor for the consultation being scheduled (e.g., credit card information, debit card information, health saving account information, or the like, as well as various combinations thereof). The payment section 561-5 may be configured to enable the patient to enter payment information or select previously entered payment information. For example, the NEW CONSULTATION REQUEST GUI 560 includes a purchase section 561-7 by which the patient may complete the purchase of the remote consultation. The purchase section 561-7 may include an indication of a service fee charged for facilitating the scheduling and execution of the consultation, a mechanism for entering promo codes, an indication of the total amount to be charged to the patient (i.e., the doctor fee and service fee less any applied promo code), one or more controls enabling the patient to agree to various terms and conditions, and a SUBMIT button which enables the patient to submit the consultation request. It will be appreciated that the NEW CONSULTATION REQUEST GUI 560 may be arranged in other ways, may include various other types of information or controls, or the like, as well as various combinations thereof.

It will be appreciated that the various graphical user interfaces of FIGS. 5A-5F provide improved user interfaces for electronic devices, improving the efficiency of use of electronic devices by users (e.g., patients, local healthcare professionals or others on behalf of patients, or the like) to perform various telemedicine-related functions (e.g., browsing doctors or other healthcare professionals, scheduling consultations with doctors or other healthcare professionals, paying for consultations with doctors or other healthcare professionals, conducting remote consultations with doctors or other healthcare professionals, consulting additional doctors or other healthcare professionals during consultations, managing and accessing health records, or the like, as well as various combinations thereof). The improved efficiency of use of electronic devices by users to perform these and other telemedicine-related functions may be provided based on use of various features of graphical user interfaces, such as menus, icons, buttons, or the like, as well as various combinations thereof.

It will be appreciated that, although primarily presented as being provided within the context of a patient application, the graphical user interfaces of FIGS. 5A-5F may be provided via a patient webpage (e.g., accessible via a web interface in which patients and healthcare professionals may access respective webpages).

It will be appreciated that the various types of information described in conjunction with the graphical user interfaces of FIGS. 5A-5F may be stored in a patient account of the patient. The patient account of the patient may be maintained by the telemedicine server 140 (e.g., on the telemedicine server 140 or a database associated with the telemedicine server 140, such as telemedicine database 141). The patient account of the patient may be encrypted or secured in other ways. The patient information in the patient account is accessible to the patient via the patient application. The patient information in the patient account, or at least a portion thereof, may be accessible to a healthcare professional (e.g., via a healthcare professional application, such as the healthcare professional application presented with respect to FIGS. 6A-6D). It is noted that a portion of the patient information in the patient account may be provided by a healthcare professional (e.g., via a healthcare professional application, such as the healthcare professional application presented with respect to FIGS. 6A-6D). The telemedicine server 140 may be configured to control and facilitate access by healthcare professionals to the patient information in the patient account (e.g., patient contact information for scheduling consultations, patient medical history information for preparing for and conducting consultations, patient medical examination information for preparing for and conducting consultations, patient payment information for obtaining payment for services, or the like, as well as various combinations thereof).

FIGS. 6A-6D depict examples of graphical user interfaces of a healthcare professional application configured for use by a healthcare professional to provide remote healthcare services to a patient.

The healthcare professional application supporting the GUIs of FIGS. 6A-6D is a user-friendly application configured to enable healthcare professionals to offer remote medical consultations for their patients. For example, the healthcare professional application supporting the GUIs of FIGS. 6A-6D may be configured to enable a healthcare professional to maintain a public profile so that patients can locate the healthcare professional, receive consultation requests from patients, review patient records and consult with patients via secure video calls, write electronic prescriptions and doctor's notes for patients, accept payment from patients electronically, and so forth. The various functions supported by the healthcare professional application may be further understood by considering the GUIs of FIGS. 6A-6D.

The healthcare professional application supporting the GUIs of FIGS. 6A-6D may be running on the healthcare professional device 122 of FIG. 1.

The GUIs of the healthcare professional application become accessible to the healthcare professional when the healthcare professional accesses the healthcare professional application.

The GUIs of the healthcare professional application include a DOCTOR GUI 610 (presented in FIG. 6A), a CONSULTATIONS GUI 620 (presented in FIG. 6B), a PATIENTS GUI 630 (presented in FIG. 6C), and a CALENDAR GUI 640 (presented in FIG. 6D).

The GUIs of the healthcare professional application each include a menu portion 609 by which the healthcare professional using the healthcare professional application may navigate between GUIs of the healthcare professional application. The menu portion 609 includes a DOCTOR button 601 for navigating to the DOCTOR GUI 610, a CONSULTATIONS button 602 for navigation to the CONSULTATIONS GUI 620, a PATIENTS button 501 for navigation to the PATIENTS GUI 630, and a CALENDAR button 604 for navigating to the CALENDAR GUI 640. The menu portion 609 also includes a SIGN OUT button 605, by which the healthcare professional may sign out of the healthcare professional application. It will be appreciated that the menu portion 609 may be arranged in other ways (e.g., a different ordering of the buttons), the menu portion 609 may include fewer or more buttons (as well as different buttons), or the like, as well as various combinations thereof.

The DOCTOR GUI 610 may be configured to support entry and presentation of personal information of the healthcare professional. The DOCTOR GUI 610 may be configured to enable the healthcare professional to enter and save a profile which will be presented to patients when searching for doctors for consultation. The DOCTOR GUI 610 includes various healthcare professional information sections 611 in which information of the healthcare professional may be presented and in which information of the healthcare professional may be added or modified. For example, the DOCTOR GUI 610 includes a basic information section 611-1. The basic information section 611-1 may include a profile picture. The basic information section 611-1 includes healthcare professional information fields specifying information such as name, address, specialties, consultation fee, associated healthcare facilities, or the like. The DOCTOR GUI 610 includes a SAVE button configured to enable the healthcare professional to save information entered into the healthcare professional information fields of the basic information section 611-1 as well as information entered into other information sections 611 as discussed below. For example, the DOCTOR GUI 610 includes an education section 611-2 in which education information of the healthcare professional may be entered and presented. The education section 611-2 may include a control element configured to support addition of education information of the healthcare professional to the education section 611-2 (e.g., selection of the control element, such as a button or other control element, results in presentation of a window or GUI via which the healthcare professional may enter education information, such as schools, degrees, certifications, or the like, as well as various combinations thereof). The education section 611-2 may include respective education elements for the respective education achievements added to the education section 611-2, each of which may include information associated with the education achievement (e.g., achievement type (e.g., degree, certification, or the like), name of the associated educational institution, dates attended, date achieved, or the like, as well as various combinations thereof) and each of which may have a control element which, when selected, enables updates to or deletion of the respective education elements. For example, the DOCTOR GUI 610 includes a medical licensure section 611-3 in which medical licensure information of the healthcare professional may be entered and presented. The medical licensure section 611-3 may include a control element configured to support addition of medical licensure information of the healthcare professional to the medical licensure section 611-3 (e.g., selection of the control element, such as a button or other control element, results in presentation of a window or GUI via which the healthcare professional may enter medical licensure information, such as licenses held, dates awarded, or the like, as well as various combinations thereof). The medical licensure section 611-3 may include respective medical licensure elements for the respective licenses added to the medical licensure section 611-3, each of which may include information associated with the respective license (e.g., license type, name of the associated educational institution, date awarded, or the like, as well as various combinations thereof) and each of which may have a control element which, when selected, enables updates to or deletion of the respective medical licensure elements. For example, the DOCTOR GUI 610 includes a professional information section 611-4 in which professional information of the healthcare professional (e.g., experience, affiliations, honors, or the like) may be entered and presented. The professional information section 611-4 may include a pair of control elements configured to support addition of professional information of the healthcare professional to the professional information section 611-4 (e.g., selection of a control element, such as a button or other control element, results in presentation of a window or GUI via which the healthcare professional may enter professional information, such as professional experiences, professional affiliations or honors, or the like, as well as various combinations thereof). The professional information section 611-4 may include respective professional information elements for the respective information added to the professional information section 611-4, each of which may include professional information associated with the healthcare professional (e.g., professional experiences, professional affiliations or honors, or the like, as well as various combinations thereof) and each of which may have a control element which, when selected, enables updates to or deletion of the respective professional information elements. For example, the DOCTOR GUI 610 includes an additional information section 611-5 in which additional information of the healthcare professional (e.g., publications, presentations, or the like) may be entered and presented. The additional information section 611-5 may include a control element configured to support addition of additional information of the healthcare professional to the additional information section 611-5 (e.g., selection of a control element, such as a button or other control element, results in presentation of a window or GUI via which the healthcare professional may enter additional information, such as publications, presentations, or the like, as well as various combinations thereof). The additional information section 611-5 may include respective additional information elements for the respective information added to the additional information section 611-5, each of which may include additional information associated with the healthcare professional (e.g., publications, presentations, or the like, as well as various combinations thereof) and each of which may have a control element which, when selected, enables updates to or deletion of the respective additional information elements. It will be appreciated that the DOCTOR GUI 610 may be arranged in other ways, may include various other types of information or controls, or the like, as well as various combinations thereof.

The CONSULTATIONS GUI 620 may be configured to support consultations by the healthcare professional with patients, enabling the healthcare professional to review scheduled consultations, review previous consultations, or the like, as well as various combinations thereof. The CONSULTATIONS GUI 620 presents each previous consultation of the healthcare professional, individually, using consultation icons 621. The consultation icon 621 for a previous consultation includes a photo of the patient consulted during that consultation, the name of the patient consulted during that consultation, a consultation status indicator (e.g., COMPLETED to indicate that the consultation was completed or OPEN to indicate that the consultation has not yet been completed), and a date that the consultation was started. It will be appreciated that the consultation icons 621 may present less or more information (including different information). The consultation icons 621 are selectable to access a CONSULTATION DETAILS GUI (omitted for purposes of clarity). The CONSULTATION DETAILS GUI may include information such as the date and time of the consultation, patient details of the doctor consulted, symptoms identified and discussed during the consultation, report orders, prescriptions, doctor's notes, or the like, as well as various combinations thereof. The CONSULTATION DETAILS GUI also may include various control elements, such as control elements by which the healthcare professional may follow-up regarding a consultation with the patient, initiate an audio or video call with the patient, or the like, as well as various combinations thereof. The CONSULTATIONS GUI 620 includes sort controls 622 by which the healthcare professional may sort the previous consultations and, thus, control the manner in which the consultation icons 621 are presented to the healthcare professional (e.g., sorting by date in ascending or descending order, sorting by patient name in ascending or descending order, or the like). The CONSULTATIONS GUI 620 includes a show past consultations control element by which the healthcare professional may request that completed consultations be hidden or displayed. It will be appreciated that the CONSULTATIONS GUI 620 may be arranged in other ways, may include various other types of information or controls, or the like, as well as various combinations thereof.

The PATIENTS GUI 630 may be configured to provide the healthcare professional with access to information about patients of the healthcare professional. The PATIENTS GUI 630 presents each patient, individually, using patient icons 631. The patient icon 631 a patient includes a photo of the patient, the date of birth of the patient, an address of the patient, or the like, as well as various combinations thereof. It will be appreciated that the patient icons 631 may present less or more information (including different information). The patient icons 631 are selectable to access a PATIENT DETAILS GUI (omitted for purposes of clarity). The PATIENT DETAILS GUI may include information such scheduled consultations, past consultations, medical history (e.g., medications, allergies, medical conditions, surgeries, social history, or the like), or the like, as well as various combinations thereof. It will be appreciated that the PATIENTS GUI 630 may be arranged in other ways, may include various other types of information or controls, or the like, as well as various combinations thereof.

The CALENDAR GUI 640 may be configured to enable the healthcare professional to review and manage his or her calendar (e.g., enter availability for consultations such that patients may schedule consultations, review scheduled consultations, or the like, as well as various combinations thereof). The CALENDAR GUI 640 may support various views, such as a day view, a week view, a month view, or the like. It will be appreciated that the CALENDAR GUI 640 may be arranged in other ways, may include various other types of information or controls, or the like, as well as various combinations thereof.

It will be appreciated that the various graphical user interfaces of FIGS. 6A-6D provide improved user interfaces for electronic devices, improving the efficiency of use of electronic devices by users (e.g., healthcare professionals or the like) to perform various telemedicine-related functions (e.g., reviewing requests for consultations from patients, scheduling consultations with patients, conducting remote consultations with patients, consulting additional doctors or other healthcare professionals during consultation, accessing patient health records, making notes and writing prescriptions, or the like, as well as various combinations thereof). The improved efficiency of use of electronic devices by users to perform these and other telemedicine-related functions may be provided based on use of various features of graphical user interfaces, such as menus, icons, buttons, or the like, as well as various combinations thereof.

It will be appreciated that, although primarily presented as being provided within the context of a healthcare professional application, the graphical user interfaces of FIGS. 6A-6D may be provided via a healthcare professional webpage (e.g., accessible via a web interface in which healthcare professionals and patients may access respective webpages).

It will be appreciated that the various types of information described in conjunction with the graphical user interfaces of FIGS. 6A-6D may be stored in a healthcare professional account of the healthcare professional. The healthcare professional account of the healthcare professional may be maintained by the telemedicine server 140 (e.g., on the telemedicine server 140 or a database associated with the telemedicine server 140, such as telemedicine database 141). The healthcare professional account of the healthcare professional may be encrypted or secured in other ways. The healthcare professional information in the healthcare professional account is accessible to the healthcare professional via the healthcare professional application. The healthcare professional information in the healthcare professional account, or at least a portion thereof, may be accessible to patients or potential patients (e.g., via a patient application, such as the patient application presented with respect to FIGS. 5A-5F). It is noted that a portion of the healthcare professional information in the healthcare professional account may be provided by patients (e.g., via a patient application, such as the patient application presented with respect to FIGS. 5A-5F). The telemedicine server 140 may be configured to control and facilitate access by patients to the healthcare professional information in the healthcare professional account (e.g., healthcare professional contact information for scheduling consultations, healthcare professional billing information for submitting payment for services, or the like, as well as various combinations thereof).

As discussed herein, the various devices, applications, and GUIs presented herein may be configured to support various processes associated with scheduling and execution of remote medical consultations of patients by healthcare professionals.

The remote healthcare capability may be configured to provide an integrated telemedicine platform that is configured to create various unique remote healthcare consultation capabilities. The integrated telemedicine platform, for a given patient to be able to obtain a remote medical consultation with a healthcare professional, may be composed of (1) the telemedicine server supporting the patient application for the patient and the healthcare professional application for the healthcare professional, including support for interaction between patient using the patient application and the healthcare professional using the healthcare professional application and (2) a telemedicine device (e.g., a telemedicine bag, a telemedicine cart, or the like) that is accessible to the patient.

The integrated telemedicine platform may be configured to support various functions which may be provided within various contexts (e.g., patient/healthcare professional interaction scenarios discussed herein or in other suitable situations). For example, functions supported by the integrated telemedicine platform may include any of the functions presented herein, such as functions for remotely connecting patients and healthcare professionals in a manner enabling remote consultations by the patients with the healthcare professionals without requiring the healthcare professionals to be in any particular location (e.g., healthcare professional call centers are not required) and without requiring patients to be in any particular location (e.g., patients can be at home, in community healthcare centers, or any other locations having telemedicine devices and being remote from the healthcare professionals), healthcare professional location functions for enabling patients to find and connect to healthcare professionals remotely, patient location functions for enabling healthcare professionals to find and connected to patients remotely, secure video calling capabilities, recording diagnostic device measurements automatically without manual data entry, remote upload of patient medical information (e.g., x-rays, MRIs, CT scans, EEGs, EKGs, blood reports, or the like) in order to provide a more detailed picture to the healthcare professional, real-time review of patient information (e.g., patient medical history, medical examination information, symptoms, or the like) while the video call is ongoing (thereby enabling the real feeling of an “in-person” type consultation), support for basic electronic medical record features (e.g., patient records, digital prescriptions, diagnostics, past medical history, doctor's notes, or the like), support for e-prescriptions (e.g., sending prescriptions electronically to the preferred pharmacy of the patient), support for follow-up consultations by the patient (e.g., with the doctor(s) of the patient, with the doctor(s) from a partner company, or the like), support for the healthcare professionals to consult specialists, support for applications that allow follow-up video visits by patients with doctors, mechanisms for patients to securely store their medical records for retrieval any time later, secure messaging between healthcare professionals and patients, mechanisms to offer continuing medical education to healthcare professionals and patients, or the like, as well as various combinations thereof.

In at least some embodiments, the integrated telemedicine platform may support remote consultation of a healthcare professional (e.g., doctor) by a patient when the patient is unable to visit any health center (e.g., at home or in another location). The patient has a telemedicine device available. The telemedicine device or a user device of the user is running the patient application to enable interaction by the patient with a healthcare professional who is using the healthcare professional application. The patients selects a healthcare professional to consult from a list of available healthcare professional or identifies a consultation previously scheduled by the patient with the healthcare professional. The healthcare professional starts a video call with the patient or the patient starts a video call with the healthcare professional. The patient uses the telemedicine device to perform a physical exam under direction from the healthcare professional. The healthcare professional examines the patient remotely, viewing the patient's body (e.g., the patient's skin, throat, nose, eyes, or the like) via video (e.g., in 1080p HD), listening to the patient's body (e.g., the patient's heart and lung sounds), and so forth. The healthcare professional gets additional patient information (e.g., pressure, SpO2, blood glucose, temperature, spirometry, or the like) as needed. The healthcare professional makes an informed judgment based on the various types of patient information and informs the patient. The healthcare professional may then write digital prescriptions, doctor's notes, or the like, as needed.

In at least some embodiments, the integrated telemedicine platform may support remote consultation of a healthcare professional (e.g., doctor) by a patient when the patient visits a community health center (CHC). The patient visits the CHC. A medical assistant (MA) at the CHC selects the healthcare professional to consult from a list of available healthcare professionals. The healthcare professional starts a video call with MA, and sees and talks to the patient. The MA uses the telemedicine device at the CHC to perform a physical exam under direction from the healthcare professional. The healthcare professional examines the patient remotely, viewing the patient's body (e.g., the patient's skin, throat, nose, eyes, or the like) via video (e.g., in 1080p HD), listening to the patient's body (e.g., the patient's heart and lung sounds), and so forth. The healthcare professional gets additional patient information (e.g., pressure, SpO2, blood glucose, temperature, spirometry, or the like) as needed. The healthcare professional makes an informed judgment based on the various types of patient information and informs the patient. The healthcare professional may then write digital prescriptions, doctor's notes, or the like, as needed.

The integrated telemedicine platform may be configured to support various other interaction types (e.g. between various other people, based on use of various other devices or applications, or the like, as well as various combinations thereof).

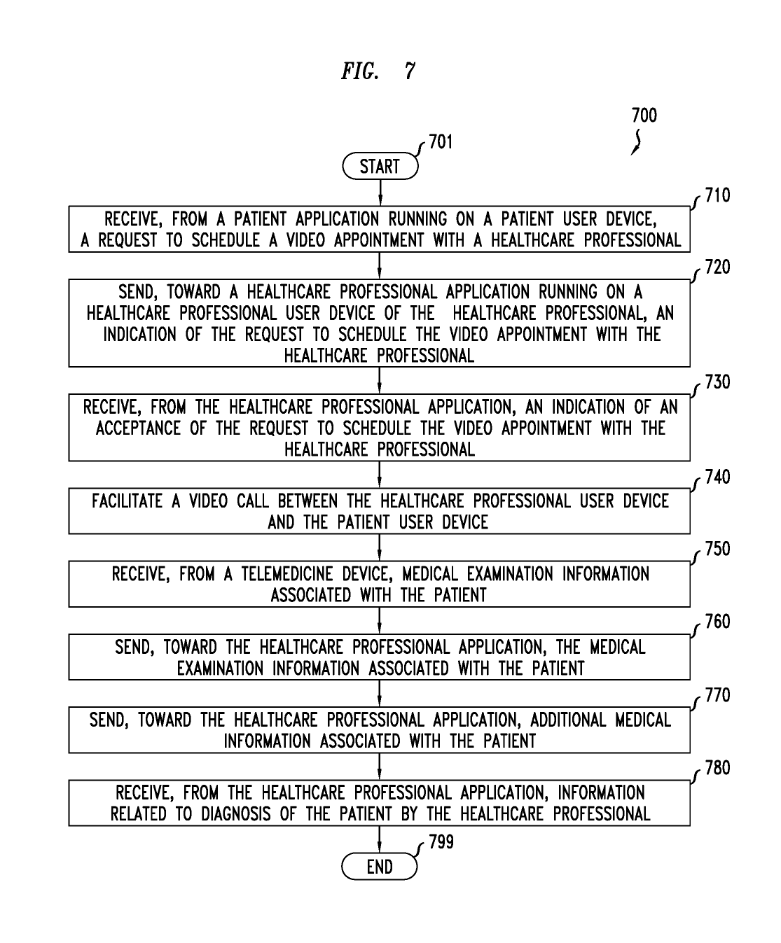

FIG. 7 depicts an example embodiment of a method for use by an integrated telemedicine platform for supporting providing of remote healthcare services to a patient. It will be appreciated that, although primarily presented herein as being performed serially, at least a portion of the functions of method 700 may be performed contemporaneously or in a different order than as presented in FIG. 7.

At block 710, receive, from a patient application running on a patient user device, a request to schedule a video appointment with a healthcare professional.

At block 720, send, toward a healthcare professional application running on a healthcare professional user device of the healthcare professional, an indication of the request to schedule the video appointment with the healthcare professional.

At block 730, receive, from the healthcare professional application, an indication of an acceptance of the request to schedule the video appointment with the healthcare professional.

At block 740, facilitate a video call between the healthcare professional user device and the patient user device.

At block 750, receive, from a telemedicine device, medical examination information associated with the patient.

At block 760, send, toward the healthcare professional application, the medical examination information associated with the patient.

At block 770, send, toward the healthcare professional application, additional medical information associated with the patient.

At block 780, receive, from the healthcare professional application, information related to diagnosis of the patient by the healthcare professional.

At block 799, method 700 ends.

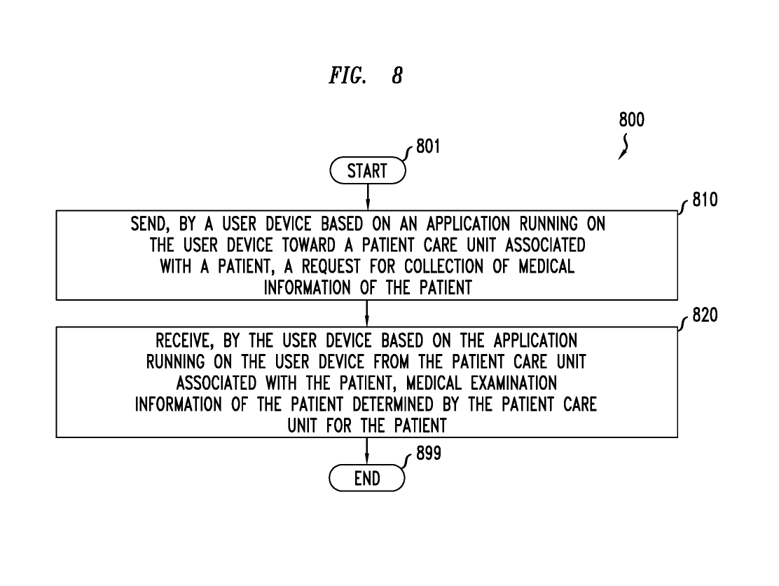

FIG. 8 depicts an example embodiment of a method for interaction by a user device with a patient care unit for supporting providing of remote healthcare services to a patient. It will be appreciated that, although primarily presented herein as being performed serially, at least a portion of the functions of method 800 may be performed contemporaneously or in a different order than as presented in FIG. 8.

At block 801, method 800 begins.

At block 810, send, by a user device based on an application running on the user device toward a patient care unit associated with a patient, a request for collection of medical information of the patient.

At block 820, receive, by the user device based on the application running on the user device from the patient care unit associated with the patient, medical examination information of the patient determined by the patient care unit for the patient.

At block 899, method 800 ends.

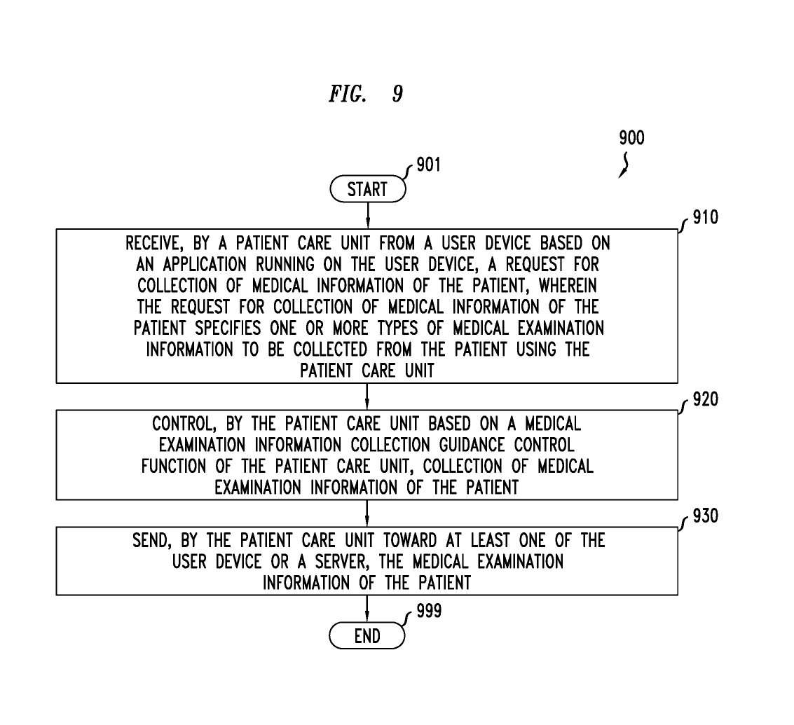

FIG. 9 depicts an example embodiment of a method for interactions by a patient care unit for supporting providing of remote healthcare services to a patient. It will be appreciated that, although primarily presented herein as being performed serially, at least a portion of the functions of method 900 may be performed contemporaneously or in a different order than as presented in FIG. 9.

At block 901, method 900 begins.

At block 910, receive, by a patient care unit from a user device based on an application running on the user device, a request for collection of medical information of the patient, wherein the request for collection of medical information of the patient specifies one or more types of medical examination information to be collected from the patient using the patient care unit.

At block 920, control, by the patient care unit based on a medical examination information collection guidance control function of the patient care unit, collection of medical examination information of the patient.

At block 930, send, by the patient care unit toward at least one of the user device or a server, the medical examination information of the patient.

At block 999, method 900 ends.

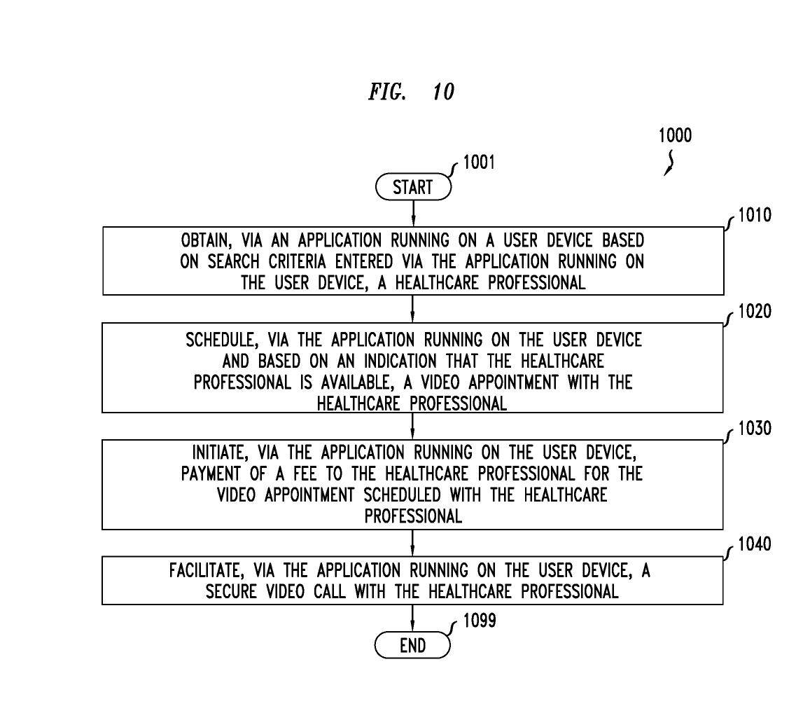

FIG. 10 depicts an example of method configured for use by a patient application at a user device of a patient. It will be appreciated that, although primarily presented herein as being performed serially, at least a portion of the functions of method 1000 may be performed contemporaneously or in a different order than as presented in FIG. 10.

At block 1001, method 1000 begins.

At block 1010, obtain, via an application running on a user device based on search criteria entered via the application running on the user device, a healthcare professional.

At block 1020, schedule, via the application running on the user device and based on an indication that the healthcare professional is available, a video appointment with the healthcare professional.

At block 1030, initiate, via the application running on the user device, payment of a fee to the healthcare professional for the video appointment scheduled with the healthcare professional.

At block 1040, facilitate, via the application running on the user device, a secure video call with the healthcare professional.

At block 1099, method 1000 ends.

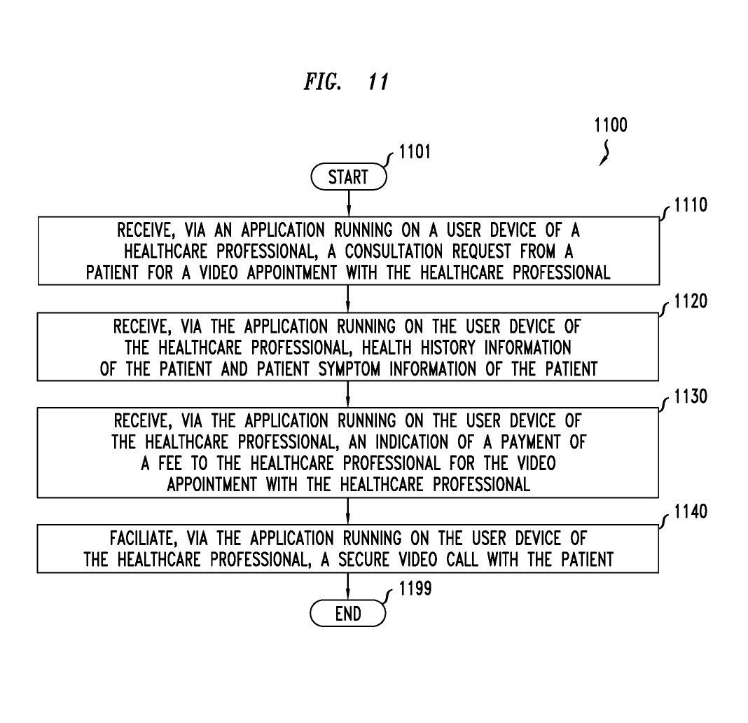

FIG. 11 depicts an example of method configured for use by a healthcare professional application at a user device of a healthcare professional. It will be appreciated that, although primarily presented herein as being performed serially, at least a portion of the functions of method 1100 may be performed contemporaneously or in a different order than as presented in FIG. 11.

At block 1101, method 1100 begins.

At block 1110, receive, via an application running on a user device of a healthcare professional, a consultation request from a patient for a video appointment with the healthcare professional.

At block 1120, receive, via the application running on the user device of the healthcare professional, health history information of the patient and patient symptom information of the patient.

At block 1130, receive, via the application running on the user device of the healthcare professional, an indication of a payment of a fee to the healthcare professional for the video appointment with the healthcare professional.

At block 1140, facilitate, via the application running on the user device of the healthcare professional, a secure video call with the patient.

At block 1199, method 1100 ends.

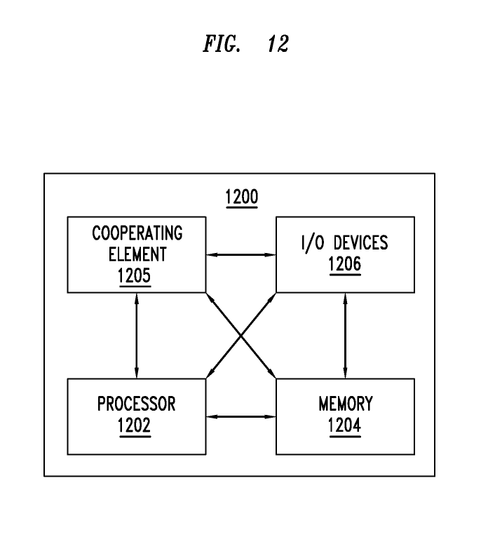

FIG. 12 depicts a high-level block diagram of a computer suitable for use in performing various functions described herein.

The computer 1200 includes a processor 1202 (e.g., a central processing unit (CPU), a processor having a set of one or more processor cores, or the like) and a memory 1204 (e.g., a random access memory (RAM), a read only memory (ROM), or the like). The processor 1202 and the memory 1204 are communicatively connected.

The computer 1200 also may include a cooperating element 1205. The cooperating element 1205 may be a hardware device. The cooperating element 1205 may be a process that can be loaded into the memory 1204 and executed by the processor 1202 to implement functions as discussed herein (in which case, for example, the cooperating element 1205 (including associated data structures) can be stored on a non-transitory computer-readable storage medium, such as a storage device or other storage element (e.g., a magnetic drive, an optical drive, or the like)).

The computer 1200 also may include one or more input/output devices 1206. The input/output devices 1206 may include one or more of a user input device (e.g., a keyboard, a keypad, a mouse, a microphone, a camera, or the like), a user output device (e.g., a display, a speaker, or the like), one or more network communication devices or elements (e.g., an input port, an output port, a receiver, a transmitter, a transceiver, or the like), one or more storage devices or elements (e.g., a tape drive, a floppy drive, a hard disk drive, a compact disk drive, or the like), or the like, as well as various combinations thereof. It will be appreciated that computer 1200 of FIG. 12 may represent a general architecture and functionality suitable for implementing functional elements described herein, portions of functional elements described herein, or the like, as well as various combinations thereof. For example, computer 1200 may provide a general architecture and functionality that is suitable for implementing one or more devices or elements presented herein, portions of one or more element presented herein, combinations of devices or elements presented herein, or the like, as well as various combinations thereof.

It will be appreciated that at least some of the functions presented herein may be implemented in software (e.g., via implementation of software on one or more processors, for executing on a general purpose computer (e.g., via execution by one or more processors) so as to provide a special purpose computer, and the like) and/or may be implemented in hardware (e.g., using a general purpose computer, one or more application specific integrated circuits (ASIC), and/or any other hardware equivalents).

It will be appreciated that at least some of the functions presented herein may be implemented within hardware, for example, as circuitry that cooperates with the processor to perform various functions. Portions of the functions/elements described herein may be implemented as a computer program product wherein computer instructions, when processed by a computer, adapt the operation of the computer such that the methods and/or techniques described herein are invoked or otherwise provided. Instructions for invoking the various methods may be stored in fixed or removable media (e.g., non-transitory computer-readable media), transmitted via a data stream in a broadcast or other signal bearing medium, and/or stored within a memory within a computing device operating according to the instructions.

It will be appreciated that the term “or” as used herein refers to a non-exclusive “or” unless otherwise indicated (e.g., use of “or else” or “or in the alternative”).

It will be appreciated that, although various embodiments which incorporate the teachings presented herein have been shown and described in detail herein, those skilled in the art can readily devise many other varied embodiments that still incorporate these teachings.

What is claimed is:

1. An apparatus, the apparatus comprising:
at least one processor; and
at least one memory including computer program code;
wherein the at least one memory and the computer program code are configured to, with the at least one processor, cause the apparatus to at least:
receive, from a patient application running on a patient user device, a request to schedule a video appointment with a healthcare professional;
send, toward a healthcare professional application running on a healthcare professional user device of the healthcare professional, an indication of the request to schedule the video appointment with the healthcare professional;
receive, from the healthcare professional application, an indication of an acceptance of the request to schedule the video appointment with the healthcare professional;
facilitate a video call between the healthcare professional user device and the patient user device;
receive, from a telemedicine device, medical examination information associated with the patient;
send, toward the healthcare professional application, the medical examination information associated with the patient;
send, toward the healthcare professional application, additional medical information associated with the patient; and
receive, from the healthcare professional application, information related to diagnosis of the patient by the healthcare professional.

2. The apparatus of claim 1, wherein the at least one memory and the computer program code are configured to, with the at least one processor, cause the apparatus to at least:
receive, from the patient application, healthcare professional filtering information;
determine, based on the healthcare professional filtering information, a list of available healthcare professionals; and
send, toward the patient application, the list of available healthcare professionals.

3. The apparatus of claim 1, wherein the indication of the request to schedule the video appointment with the healthcare professional includes symptom information of the patient and payment information of the patient.

4. The apparatus of claim 1, wherein the at least one memory and the computer program code are configured to, with the at least one processor, cause the apparatus to at least:
receive, from the healthcare professional application prior to the video call, a request for patient information associated with the patient; and
determine whether the healthcare professional is authorized to access the patient information associated with the patient; and
send, toward the healthcare professional application based on a determination that the healthcare professional is authorized to access the patient information associated with the patient, the patient information associated with the patient.

5. The apparatus of claim 4, wherein the patient information includes at least one of medical history information of the patient, symptom information of the patient, or past medical examination information of the patient.

6. The apparatus of claim 1, wherein the at least one memory and the computer program code are configured to, with the at least one processor, cause the apparatus to at least:
receive, from the healthcare professional application, a request for the medical examination information associated with the patient; and
send, toward the telemedicine device, a message indicative of the request for the medical examination information associated with the patient.

7. The apparatus of claim 6, wherein the message indicative of the request for the medical examination information associated with the patient is configured to cause the telemedicine device to prompt an operator of the telemedicine device to initiate collection of the medical examination information associated with the patient.

8. The apparatus of claim 6, wherein the message indicative of the request for the medical examination information associated with the patient is configured to cause the telemedicine device to initiate a series of medical examination information collection actions configured to collect respective types of medical examination information.

9. The apparatus of claim 1, wherein the medical examination information includes at least one of video of a portion of a body of the patient, audio of a portion of the body of the patient, a temperature of the patient, a blood pressure reading of the patient, a pulse-ox reading of the patient, a blood glucose reading of the patient, or a spirometry reading of the patient.

10. The apparatus of claim 1, wherein the at least one memory and the computer program code are configured to, with the at least one processor, cause the apparatus to at least:
receive, from the healthcare professional application, a request for the additional medical information; and
send the additional medical information toward the healthcare professional application based on the request for the additional medical information.

11. The apparatus of claim 1, wherein the additional medical information includes at least one of an x-ray, a magnetic resonance image (MRI), a computed tomography (CT) scan, an electrocardiogram (EKG), or a blood report.

12. The apparatus of claim 1, wherein the information related to diagnosis of the patient by the healthcare professional includes at least one of a diagnosis description to be associated with an account of the patient, a prescription, or a note.

13. The apparatus of claim 1, wherein the information related to diagnosis of the patient by the healthcare professional includes a prescription, wherein the at least one memory and the computer program code are configured to, with the at least one processor, cause the apparatus to at least:
determine a preferred pharmacy of the patient; and
send the prescription toward a device of the preferred pharmacy of the patient.

14. A method, comprising:
receiving, from a patient application running on a patient user device, a request to schedule a video appointment with a healthcare professional;
sending, toward a healthcare professional application running on a healthcare professional user device of the healthcare professional, an indication of the request to schedule the video appointment with the healthcare professional;
receiving, from the healthcare professional application, an indication of an acceptance of the request to schedule the video appointment with the healthcare professional;
facilitating a video call between the healthcare professional user device and the patient user device;
receiving, from a telemedicine device, medical examination information associated with the patient;
sending, toward the healthcare professional application, the medical examination information associated with the patient;
sending, toward the healthcare professional application, additional medical information associated with the patient; and
receiving, from the healthcare professional application, information related to diagnosis of the patient by the healthcare professional.

15. A non-transitory computer readable medium comprising program instructions for causing an apparatus to at least:
receive, from a patient application running on a patient user device, a request to schedule a video appointment with a healthcare professional;
send, toward a healthcare professional application running on a healthcare professional user device of the healthcare professional, an indication of the request to schedule the video appointment with the healthcare professional;
receive, from the healthcare professional application, an indication of an acceptance of the request to schedule the video appointment with the healthcare professional;
facilitate a video call between the healthcare professional user device and the patient user device;
receive, from a telemedicine device, medical examination information associated with the patient;
send, toward the healthcare professional application, the medical examination information associated with the patient;
send, toward the healthcare professional application, additional medical information associated with the patient; and
receive, from the healthcare professional application, information related to diagnosis of the patient by the healthcare professional.

16. A patient care unit, comprising:
a portable housing including:
a first portion, the first portion including a computing device having a touch-sensitive display, a microphone, a speaker, and a set of communication interfaces;
a second portion configured to provide a base for the portable housing, the second portion including a set of receptacles configured to house a set of medical instruments, the set of medical instruments including a set of wired medical instruments and a set of wireless medical instruments;
a hinge pivotally connecting the first portion to the second portion to permit the first portion to pivot with respect to the second portion between a closed position and an open position; and
a wired medical instrument interface control element communicatively connected to the computing device;
the wired medical instrument interface control element configured to control interfacing between the wired medical instruments and the computing device;
the set of communication interfaces including a local wireless communication interface configured to support interfacing between the wireless medical instruments and the computing device.

17. The patient care unit of claim 16, wherein the set of wired medical instruments includes at least one of a digital stethoscope, an examination camera, a digital otoscope, or a digital iriscope.

18. The patient care unit of claim 16, wherein the wired medical instrument interface control element includes a set of communication interfaces configured to support wired connection of the respective wired medical instruments.

19. The patient care unit of claim 16, wherein the wired medical instrument interface control element includes a communication interface configured to support a wired connection of the wired medical instrument interface control element to the computing device.

20. The patient care unit of claim 16, wherein the wired medical instrument interface control element includes a set of buttons associated with the respective wired medical instruments.

21. The patient care unit of claim 16, wherein the wired medical instrument interface control element includes a button associated with one of the wired medical instruments, wherein the wired medical instrument interface control element is configured to, responsive to detection of an actuation of the button, perform a control action associated with the one of the wired medical instruments.

22. The patient care unit of claim 21, wherein the control action associated with the one of the medical instruments includes at least one of:
initializing the one of the medical instruments to enter a state in which the one of the medical instruments is configured to take a reading; or
initializing the computing device to enter a state in which the computing is configured to receive a reading from the one of the medical instruments.

23. The patient care unit of claim 16, wherein the set of wireless medical instruments includes at least one of a blood pressure monitor, a pulse oximeter, a glucometer, a spirometer, or a thermometer.

24. The patient care unit of claim 16, wherein the computing device includes a set of communication interfaces configured to support wireless connection of the respective wireless medical instruments to the computing device.

25. The patient care unit of claim 16, wherein the computing device is configured to support display of a wireless medical instrument interface control element including a set of graphical objects associated with the respective wireless medical instruments.
